(19) United States
(12) Patent Application Publication
(10) Pub. No.: US 2026/0117397 A1
(43) Pub. Date: Apr. 30, 2026

(54) PROPANE DEHYDROGENATION PROCESS AND POLYPROPYLENE MANUFACTURING PROCESS

(71) Applicant: TOTALENERGIES ONETECH, Courbevoie (FR)

(72) Inventors: Julien GRAND, Seneffe (BE); Marc RICHET, Seneffe (BE); José SERRA, Valencia (ES); Maria VALLS ESTEVE, Valencia (ES); Jesus BERNAD, Valencia (ES)

(21) Appl. No.: 19/483,318

(22) PCT Filed: Jun. 13, 2024
(86) PCT No.: PCT/EP2024/066440

§ 371 Date: Nov. 11, 2025

(30) Foreign Application Priority Data
Jun. 14, 2023 (EP) 23315244.6
Feb. 29, 2024 (EP) 24315072.9

(51) Int. Cl. C25B 3/03; B01J 8/00; B01J 8/02; C07C 5/32; C08F 10/06; C25B 1/02; C25B 9/23; C25B 15/08

(52) U.S. Cl. CPC C25B 3/03; B01J 8/005; B01J 8/0278; C07C 5/324; C08F 10/06; C25B 1/02; C25B 9/23; C25B 15/081; C25B 15/085

(57) ABSTRACT

The disclosure concerns a propane dehydrogenation process, remarkable in that it comprises the steps of (a) providing a stream comprising at least propane; (b) providing at least one proton-conducting catalytic membrane, each proton-conducting catalytic membrane comprising an anode, an electrolyte layer disposed on top of the anode and a porous cathode disposed on top of the electrolyte layer, wherein the anode comprises at least one dehydrogenation catalyst; (c) feeding within the anode of said one or more proton-conducting catalytic membranes under propane dehydrogenation conditions the stream provided at step (a); and (d) recovering a first effluent comprising at least propylene. The disclosure also concerns a polypropylene manufacturing process and an installation to conduct said process.

### TECHNICAL FIELD

The present disclosure relates to a propane dehydrogenation process and to a polypropylene manufacturing process, as well as to an installation to conduct said polypropylene manufacturing process.

### TECHNICAL BACKGROUND

During a polypropylene manufacturing process, in addition to the formation of polypropylene from the polymerization of propylene, a significant amount a propane is present and does not react. In a dedicated separation zone, it is possible to separate the polypropylene, and retrieve a stream comprising unreacted propylene and propane and optionally a stream comprising the solvent into which the polymerization has taken place. The stream comprising unreacted propylene and propane is then sent to a C3 splitter to produce an overhead having a propylene content of at least 99.5 wt. % based on the total weight of said overhead and then is recycled to the polymerization zone. Upon splitting, a rich-propane bottom stream is also produced. This rich-propane bottom stream can comprise propylene up to 40 wt. % based on the total weight of the said bottom stream. In the disclosure EP2807195, this rich-propane bottom stream is further separated thanks to a membrane separation zone into a permeate with an enhanced propylene content that can be recycled to the polymerization zone, and a purge with an enhanced propane content. Nevertheless, this kind of polypropylene manufacturing process represents an economical loss since a part of the starting stream is not transformed into the desired product (i.e., polypropylene), but in propane that has to be removed. The removal of the propane, using both the C3 splitter and the membrane separation zone, results in a polypropylene manufacturing process that is not energetically efficient for such a process on large scale and in the long term.

Although the propane could be dehydrogenated back into propylene that could thus be recycled to the polymerization zone, such dehydrogenation reactions are not straightforward and cannot be implemented easily.

Dehydrogenation reactions are indeed carried out without hydrogen extraction, limiting therefore the industrial processes to about 50% conversion. In addition, such reactions lead to significant and fast coking of the catalyst. Industrial processes require therefore either continuous decoking (with H₂ or water). H₂ produced must be separated from the product stream after the reaction.

Concerning the dehydrogenation aspect, a solution was brought by EP2534721 and/or by WO2011098525 to use a tubular reactor comprising a first zone comprising a dehydrogenation catalyst and a second zone separated from said first zone by a proton-conducting membrane comprising forming a layered structure of a porous and non-porous mixed metal oxide as a support and a dehydrogenation catalyst that is deposited on the membrane and that can adhere to it or freely lie on it. Similar proton-conducting ceramic membranes are described in WO2014187978.

In US20200248321, a membrane reactor for conducting the dehydrogenation of alkanes to alkenes presents one or more tubes lined with one or more reactive ceramic membranes or layered throughout the one or more tubes. The membrane reactor allows a heated feed of alkanes to come into contact with the one or more ceramic membranes, wherein an alkane would react therewith, allowing alkenes to continue to flow through the one or more tubes and out of the system in reactor effluent feed, while hydrogen generated from the reaction of the alkane with the membrane would pass through the membrane and be physically separated.

However, it appeared that the manufacturing of defect-free straight tubes is a critical issue for further development of the dense ion-conducting membrane propane-to-propylene on-purpose technology. Defects in the membrane enhance coke formation with the result that the membrane was destroyed. The fragility of the membranes is also an issue.

The objective of this disclosure is therefore to provide a propane dehydrogenation process that performs with high conversion. In particular, the objective of this disclosure is to provide a polypropylene manufacturing process which is more economically and energetically efficient.

### SUMMARY

According to a first aspect, the disclosure relates to a propane dehydrogenation (PDH) process, said process is remarkable in that it comprises the following steps
a) providing a stream comprising at least propane;
b) providing at least one proton-conducting catalytic membrane, each proton-conducting catalytic membrane comprising an anode, an electrolyte layer disposed on top of the anode and a porous cathode disposed on top of the electrolyte layer, wherein the anode comprises one or more channels, each of said channels comprising at least one dehydrogenation catalyst; wherein said one or more channels are flared depth channels or rectangular channels, and wherein said one or more channels are made in copper or steel;
c) feeding within the anode of said one or more proton-conducting catalytic membranes under propane dehydrogenation conditions the stream provided at step (a);
d) recovering a first effluent comprising at least propylene;
e) optionally, recovering a second effluent comprising hydrogen.

Surprisingly, as the proton-conducting catalytic membrane of the present disclosure consists in integrating one or more dehydrogenation catalysts within an anode acting as support layer of a proton-conducting catalytic membrane and therefore within the proton-conducting catalytic membrane itself, and not only on its top, this results in a favourable extraction of the hydrogen by expanding the surface contact between the one or more dehydrogenation catalysts and the membrane. In fine, this has the beneficial effect of displacing the chemical equilibrium towards the products during a dehydrogenation reaction according to Le Chatelier's principle and subsequently enhancing the conduct of the propane dehydrogenation (PDH) process by providing among other a conversion superior to 40%, preferably superior to 50%.

For example, the disclosure relates to a propane dehydrogenation (PDH) process, said process is remarkable in that it comprises the following steps
a) providing a stream comprising at least propane;
b) providing at least one proton-conducting catalytic membrane, each proton-conducting catalytic membrane comprising an anode, an electrolyte layer disposed on top of the anode and a porous cathode disposed on top of the electrolyte layer, wherein the anode comprises one or more channels, each of said channels comprising at least one dehydrogenation catalyst; wherein said one or more channels are flared depth channels, and wherein said one or more flared depth channels are made in copper or steel;
c) feeding within the anode of said one or more proton-conducting catalytic membranes under propane dehydrogenation conditions the stream provided at step (a);
d) recovering a first effluent comprising at least propylene;
e) optionally, recovering a second effluent comprising hydrogen.

Advantageously, the propane dehydrogenation conditions of step (c) comprise a temperature ranging between 425° C. and 575° C.; preferably between 430° C. and 570° C., more preferably between 435° C. and 565° C.

For example, the propane dehydrogenation conditions comprise a space velocity of at least 150 Nml/h/g, or of at least 200 Nml/h/g, or of at least 250 Nml/h/g, or of at least 300 Nml/h/g, or of at least 350 Nml/h/g, or of at least 400 Nml/h/g, or of at least 450 Nml/h/g, or of at least 500 Nml/h/g. For example, the propane dehydrogenation conditions comprise a space velocity ranging between 150 Nml/h/g and 1000 Nml/h/g, or between 200 Nml/h/g and 1000 Nml/h/g, or between 250 Nml/h/g and 1000 Nml/h/g, or between 300 Nml/h/g and 1000 Nml/h/g, or between 350 Nml/h/g and 1000 Nml/h/g, or between 400 Nml/h/g and 1000 Nml/h/g, or between 450 Nml/h/g and 1000 Nml/h/g, or between 500 Nml/h/g and 1000 Nml/h/g.

In a preferred embodiment, the stream provided at step (a) further comprises steam. With preference, the amount of steam is ranging between 1 vol. % and 15 vol. % based on the total volume of the stream provided at step (a); more preferably ranging between 2 vol. % and 10 vol. %.

For example, the process further comprises the step of providing steam into the stream provided at step (a). Said step of providing steam is performed continuously or in a pulsed manner (namely at intervals), preferably in a pulsed manner.

Advantageously, in said preferred embodiment, the one or more proton-conducting catalytic membranes provided at step (b) are one or more co-ionic catalytic membranes.

According to a second aspect, the disclosure relates to a polypropylene manufacturing process, said process comprising the following steps:
a) providing a first propylene stream, said first propylene stream optionally comprising one or more solvents;
b) polymerizing the first propylene stream under polymerization conditions to form an effluent comprising polypropylene and at least propylene and propane and optionally the one or more solvents if any;
c) separating the effluent to generate a first stream comprising polypropylene and a second stream comprising at least propylene and propane and optionally a third stream comprising the one or more solvents if any;
d) optionally, recycling the third stream if any;
f) splitting the second stream to produce an overhead comprising propylene and a purge stream, said purge stream being a propane-rich stream further comprising propylene;
g) optionally, recycling the overhead into the first propylene stream provided at step (a);
   wherein the process is remarkable in that it further comprises a first dehydrogenating step (h) and/or a second dehydrogenating step (e); wherein said first dehydrogenating step (h) is the step of dehydrogenating the purge stream produced at step (f) to enrich in propylene the purge stream; wherein said second dehydrogenating step (e) is carried out after the separation step (c) and before the splitting step (f) and is the step of dehydrogenating the second stream generated at step (c) to enrich in propylene the second stream; and wherein said first and second dehydrogenating steps are each a propane dehydrogation (PDH) step as defined in accordance with the first aspect.

With preference, said process comprises a first dehydrogenating step (h).

Surprisingly, it was found that it is possible to recycle a significant amount a propane that is produced during a polypropylene manufacturing process, rendering it then more economically and energetically viable. Thus, the first dehydrogenating step (h), which is the the dehydrogenation of the purge stream being a propane-rich stream, using a proton-conducting catalytic membrane, further increases the amount of produced propylene.

In addition, a second dehydrogenating step (e), using a proton-conducting catalytic membrane, can be undertaken on the stream comprising at least (unreacted) propylene and propane.

The proton-conducting catalytic membrane consists in integrating one or more dehydrogenation catalysts within an anode which forms the support layer of the proton-condcuting catalytic membrane and therefore within the proton-conducting catalytic membrane itself, and not only on its top, resulting in a favourable extraction of the hydrogen by expanding the surface contact between the one or more dehydrogenation catalysts and the membrane. In fine, this has the beneficial effect of displacing the chemical equilibrium towards the products during a dehydrogenation reaction according to Le Chatelier's principle, thus favouring the transformation of propane back to propylene.

When this dehydrogenation step occurs before the splitting step and thus on the second stream comprising propylene and propane, namely during the second dehydrogenating step (e), the splitting step itself is facilitated. Indeed, the second dehydrogenating step (e) reduces the quantity of propane and increases the quantity of propylene. The energy-demanding splitter duty is reduced as the splitter has then less propane to remove when the second stream is dehydrogenated during the second dehydrogenating step (e).

Advantageously, the second stream comprises after the second dehydrogenating step (e) at least 1.5 times more propylene than before said second dehydrogenating step (e). For example, the second stream comprises after the second dehydrogenating step (e) at least 50 vol. % of propylene based on the total volume of the second stream, preferably at least 60 vol. %, more preferably at least 70 vol. %, even more preferably at least 80 vol. %, most preferably at least 90 vol. %, even most preferably at least 95 vol. %.

Advantageously, the second stream further comprises after the second dehydrogenating step (e) propane, ethylene, ethane, methane, or a mixture thereof. For example, the second stream further comprises, after the second dehydrogenating step (e) and in a total amount inferior to 30 vol. % of the second stream based on the total volume of said second stream, propane, ethylene, ethane, methane, or a mixture thereof; preferably inferior to 25 vol. %, more preferably inferior to 20 vol. %, even more preferably inferior to 15 vol. %, most preferably inferior to 10 vol. %, even most preferably inferior to 5 vol. %, or inferior to 2.5 vol. %, or inferior to 1 vol. %.

Advantageously, the purge stream comprises after the first dehydrogenating step (h) at least 1.5 times more propylene than before said first dehydrogenating step (h).

Advantageously, during the first dehydrogenating step (h) and/or during the second dehydrogenating step (e), a step of recovering a hydrogen effluent is carried out. This provides the advantage that the polypropylene manufacturing process produces not only the expected polypropylene in a more efficient way since the significant amount of propane can be now reconverted in propylene which is the starting material of the present presence but also produces hydrogen that can be used as an energy source for downstream processes.

For example, the propane dehydrogenation conditions of the first dehydrogenating step (h) and/or the second dehydrogenating step (e) comprise one or more of the following:
providing an electrical current between the anode and the porous cathode of the one or more proton-conducting catalytic membranes. With preference, said electrical current has a density of at least 0.10 A/cm² as measured by ampere meter/power supply instrument and divided by membrane surface or calculated from conductivity measurements (from electrochemical impedance spectroscopy, EIS), more preferably of at least 0.15 A/cm², even more preferably of at least 0.20 A/cm², most preferably of at least 0.25 A/cm², even most preferably of at least 0.30 A/cm², or of at least 0.35 A/cm². For example, said electrical current has a density ranging between 0.10 A/cm² and 0.75 A/cm² as measured by ampere meter/power supply instrument and divided by membrane surface or calculated from conductivity measurements (from electrochemical impedance spectroscopy, EIS), more preferably between 0.15 A/cm² and 0.70 A/cm², even more preferably between 0.20 A/cm² and 0.65 A/cm², most preferably between 0.25 A/cm² and 0.60 A/cm², or between 0.25 A/cm² and 0.55 A/cm², or between 0.25 A/cm² and 0.50 A/cm², or between 0.30 A/cm² and 0.45 A/cm². With preference, said electrical current has electric potential ranging between 0.4 V and 1.8 V as measured by voltmeter/power supply instrument, more preferably between 0.5 V and 1.0 V.
a temperature ranging between 400° C. and 700° C., preferably between 450° C. and 650° C., more preferably between 500° C. and 600° C., even more preferably between 510° C. and 590° C., most preferably between 520° C. and 580° C., even most preferably between 525° C. and 575° C., or between 530° C. and 570° C.
a pressure ranging between 0.01 MPa and 1 MPa, preferably between 0.05 MPa and 0.9 MPa, more preferably between 0.09 MPa and 0.8 MPa, or between 0.1 MPa and 0.6 MPa.
a space velocity ranging between 100 NmL/h/g and 800 NmL/h/g as measured by the flowrate from floweter divided by the catalyst mass, preferably between 200 NmL/h/g and 700 NmL/h/g, more preferably between 300 NmL/h/g and 600 NmL/h/g.
a space velocity higher than 550 Nml/h/g, and a temperature ranging between 550° C. and 650° C., preferably a temperature ranging between 550° C. and 600° C.
a space velocity lower than 350 Nml/h/g, and a temperature ranging between 500° C. and 650° C., preferably a temperature ranging between 500° C. and 600° C.

For example, the second stream has during the second dehydrogenating step (e) a feed flow ranging between 20 T/h and 40 T/h as measured by a flowmeter, preferably between 22 T/h and 38 T/h, or between 24 T/h and 36 Th/h.

For example, the purge stream has during the first dehydrogenating step (h) a feed flow ranging between 0.5 T/h and 5 T/h as measured by a flowmeter, preferably between 1 T/h and 4 T/h, or between 1.5 T/h and 3.5 T/h.

For example, step (b) is carried out in a liquid phase or a gas phase.

For example, step (b) comprises providing a Ziegler-Natta catalyst or a metallocene catalyst.

For example, step (c) is carried out by flashing or by cyclonic separation.

According to a third aspect, the disclosure relates to Installation to conduct a polypropylene manufacturing process as defined in accordance with the first aspect, said installation comprising one polymerization zone with one polymerization reactor and one separation zone downstream of said polymerization zone, said separation zone comprising a polymer separation apparatus and a splitter with a bottom outlet, wherein said installation further comprises a first line between said polymerization reactor and said polymer separation apparatus, said installation is remarkable in that it comprises one or more first dehydrogenation reactors arranged downstream of the bottom outlet of the splitter and/or one or more second dehydrogenation reactors arranged upstream of the splitter; each first and second dehydrogenation reactor comprising at least one proton-conducting catalytic membrane, each proton-conducting catalytic membrane comprising an anode, an electrolyte layer disposed on top of the anode and a porous cathode disposed on top of the electrolyte layer, and wherein the anode comprises one or more channels, each of said channels comprising at least one dehydrogenation catalyst, wherein said one or more channels are flared depth channels or rectangular channels, and wherein said one or more channels are made in copper or steel.

With preference, said installation comprises one or more first dehydrogenation reactors arranged downstream of the bottom outlet of the spliter.

With preference, said one or more dehydrogenation reactors are within the separation zone.

Advantageously, said installation further comprises one scrubber arranged upstream of each first and/or second dehydrogenation reactor.

Advantageously, the splitter has a top outlet, and the installation further comprises a recycling line between the top outlet of the spliter and the polymerization reactor. With preference, a dryer system is upstream the polymerization reactor on the recycling line.

Advantageously, each first and/or second dehydrogenation reactor has a planar geometry. It has been indeed noticed that when the dehydrogenation reactor module is designed to reduce gas polarization, by being planar, it is still possible to improve the extracting capability of the proton-conducting catalytic membrane that is described above. Such dehydrogenation reactor further minimizes coking formation and therefore preserves the activity of the one or more dehydrogenation catalysts.

Advantageously, said reactor has an inlet and an outlet, and said reactor is remarkable in that the inlet is in a first zone arranged so that a feed to said reactor via the inlet contacts the one or more dehydrogenation catalysts of the proton-conducting catalytic membrane before passing to the outlet, the outlet being for example in a second zone that is downstream of the proton-conducting catalytic membrane. With preference, the inlet has surface area identical to the surface area of the outlet or the inlet has a surface area smaller than the surface area of the outlet. More preferably, the inlet has a surface area smaller than the surface area of the outlet.

Advantageously, said reactor has an inlet and an outlet, and said reactor is remarkable in that the electroconductive layer of the one or more proton-conducting catalytic membranes comprises one or more channels, each of said channels comprising the one or more dehydrogenation catalysts, and in that the inlet is in a first zone arranged so that a feed to said reactor via the inlet is conducted through the one or more channels and contacts the one or more dehydrogenation catalysts before passing to the outlet being for example in a second zone that is downstream of the proton-conducting catalytic membrane. With preference, the inlet has surface area identical to the surface area of the outlet or the inlet has a surface area smaller than the surface area of the outlet. More preferably, the inlet has a surface area smaller than the surface area of the outlet.

With preference, the inlet and outlet of the reactor are arranged on faces of the reactor that are opposed to each other. In other words, the inlet is in a first zone arranged so that a feed to said reactor via the inlet is conducted towards the proton-conducting catalytic membrane and the outlet is in a second zone that is downstream of the proton-conducting catalytic membrane. With preference, the first zone and the second zone each form one face of the reactor and are advantageously opposed to each other. Alternatively, the first zone and the second zone are arranged on a single face of the reactor.

The following features advantageously further defines the one or more proton-conducting catalytic membranes of each dehydrogenation reactor:
The anode comprises one or more channels arranged in parallel to each other, each of said channels comprising the one or more dehydrogenation catalysts. For example, at least a part of said one or more channels are rectangular channels, flared depth channels, flared width channels, bended channels, or corrugated channels; more preferably, all of said one or more channels are rectangular channels or flared depth channels; even more preferably, all of said one or more channels are flared depth channels.
The proton-conducting catalytic membrane presents a hydrogen extraction ratio ranging between 0.20 and 0.99, or between 0.25 and 0.90, at a temperature of at least 500° C. and/or at a pressure of at most 0.1 MPa, the hydrogen extraction ratio being simulated by computational fluid dynamics (CFD) and/or quantified by gas chromatography (GC) during the reaction; with preference, the proton-conducting catalytic membrane presents a hydrogen extraction ratio ranging between 0.20 and 0.99, or between 0.25 and 0.90, at a temperature of at least 550° C. and/or at a pressure of at most 0.05 MPa.
The anode is made of one or more first metals and/or of one or more spinels. With preference, the one or more first metals are selected from Cu, Fe, Cr, Ag, Ni, Mo, Pt, Pd, Au, Mn or any mixtures thereof, more preferably from Cu, Fe, Cr, Ag, Ni, Mo, Pt, Pd or any mixtures thereof, even more preferably from Cu, Fe, Cr, Ag, Ni, Mo or any mixtures thereof, most preferably from Cu, Fe, Cr, Ag, Ni or any mixtures thereof, such as Cu, Fe, Cr, Ni or any mixtures thereof, or such as Cu, Fe, Cr or any mixtures thereof, or such as Cu and/or Ag. For example, the one or more first metals and/or the one or more spinels are doped with one or more dopants, preferably selected from Cu, Li, Cr or a mixture thereof.
The anode is made of or comprises steel. For example, the anode is made of or comprises stainless steel, more preferably ferritic stainless steel. For example, the steel comprises between 60 wt. % and 80 wt. % of iron based on the total weight of the steel, preferably between 63 wt. % and 75 wt. % of iron. For example, the steel comprises between 11 wt. % and 18 wt. % of chromium based on the total weight of the steel, preferably between 12 wt. % and 17 wt. %. For example, the steel comprises between 10 wt. % and 14 wt. % of nickel based on the total weight of the steel, preferably between 11 wt. % and 13 wt. %. For example, the steel comprises between 1 wt. % and 3 wt. % of molybdenum. For example, the steel comprises at least 60 wt. % of iron and between 11 wt. % and 18 wt. % of chromium based on the total weight of the steel. With preference, the steel also comprises less than 0.15 wt. % of carbon based on the total weight of steel; preferably less than 0.12 wt. %. For example, the steel comprises between 60 wt. % and 80 wt. % of iron, between 11 wt. % and 18 wt. % of chromium and less than 0.15 wt. % of carbon, based on the total weight of the steel.
The anode is devoid of pores.
The one or more dehydrogenation catalysts comprise one or more metal-based catalysts, one or more metal oxide-based catalysts, one or more zeolites, one or more transition metal carbides, one or more transition metal nitrides, one or more carbon nanotubes, or any mixtures thereof. With preference, the one or more dehydrogenation catalysts comprise one or more metal-based catalysts, one or more metal oxide-based catalysts, one or more zeolites, or any mixtures thereof. More preferably, the one or more dehydrogenation catalysts comprise one or more metal-based catalysts. For example, the one or more zeolites are selected from the group of CHA, MFI families or any mixtures thereof. For example, the one or more zeolites are doped with one or metals selected from Mo, W, Fe, V, Cr or any mixtures thereof. For example, the one or more metal-based catalysts and/or the one or more metal oxide-based catalyst comprises one or more metals selected from Pd, Pt, Sn, Mo, W, Fe, V, Cr or any mixtures thereof.
The electrolyte layer is or comprises one or more perovskite materials with one or more cations selected from Ba, Ce, Zr, Y, La, Sr, Mn, Fe, Eu, Tb, Ni, Yb, Pr, Sm, Zn, Gd, Er, Co, Nd, or any mixtures thereof; with preference, said one or more cations are selected from Ba, Ce, Zr, Y, Ni, or any mixtures thereof; more preferably said one or more cations are selected from Ba, Ce, Zr, Y or any mixtures thereof.
The electrolyte layer is or comprises one or more perovskite materials with an electrical conductivity ranging between 10⁻⁴ S/cm and 10⁻³ S/cm as determined by electrochemical impedance spectroscopy measurements performed at 600° C. under an atmosphere comprising Ar and H₂ in a ratio 75/25.
The anode has a thickness ranging between 3 mm and 6 mm as designed by SolidWorks software, preferably between 3.5 mm and 5.5 mm.
The electrolyte layer has a thickness ranging between 20 μm and 40 μm as determined by scanning electron microscopy, preferably between 25 μm and 35 μm.
The porous cathode has a thickness ranging between 30 μm and 50 μm as determined by scanning electron microscopy, preferably between 35 μm and 45 μm.
The porous cathode comprises a mixture of one or more electrolytes and one or more second metals. For example, the one or more second metals are selected from Ni, Fe, Co, Pd, Pt, or any mixtures thereof, more preferably Ni. With preference, the amount of the one or more second metals is ranging between 50 wt. % and 70 wt. % of the total weight of said mixture, more preferably between 55 wt. % and 65 wt. %. For example, the amount of nickel is ranging between 50 wt. % and 70 wt. % of the total weight of said mixture, more preferably between 55 wt. % and 65 wt. %.
The porous cathode is a macroporous layer.
The proton-conducting catalytic membrane is a co-ionic catalytic membrane.

In an embodiment, the dehydrogenation reactor comprises an arrangement of at least two proton-conducting catalytic membranes arranged on top of each other, preferably an arrangement of at least three, or of at least four, or of at least five.

In another complementary and/or alternative embodiment, the dehydrogenation reactor comprises an arrangement of at least two proton-conducting catalytic membranes which are coplanar with each other and/or adjacent with each other.

Advantageously, whichever the embodiment selected or when said dehydrogenation reactor comprises more than one proton-conducting catalytic membrane, said dehydrogenation reactor further comprises a spacer between each proton-conducting membrane. For example, the spacer is an electro-conducting plate, preferably a steel plate, more preferably a stainless steel plate, even more preferably a ferritic stainless steel plate.

According to a fourth aspect, the disclosure relates to a method for making a proton-conducting membrane, remarkable in that said method comprises the following steps:
a) providing a mixture of one or more electrolytes and one or more second metals to form a solution of mixed oxides;
b) pressing uniaxially the mixture provided at step (a) to form a porous cathode;
c) optionally, calcining said porous cathode
d) providing one or more electrolytes;
e) screen-printing said one or more electrolytes on the porous cathode or on the calcined porous cathode if step (c) is carried out, to obtain a porous cathode with an electrolyte layer;
f) sintering said porous cathode with an electrolyte layer;
g) providing an anode made of one or more first metals and/or of one or more spinels;
i) coating said anode provided at step (g) on the electrolyte layer of the sintered porous cathode with an electrolyte layer obtained at step (f), so as to obtain a proton-conducting membrane.

According to a fifth aspect, the disclosure relates to a method for making a proton-conducting catalytic membrane as defined above, remarkable in that said method comprises a method for making a proton-conducting membrane as defined in the fourth aspect, and in that the method for making a proton-conducting membrane as defined in the fourth aspect further comprises after the step (g), the step (h) of depositing at least one dehydrogenation catalyst on said anode provided at step (g), so as to form a catalytic anode, and wherein the step (i) is the step of assembling together the sintered porous cathode with an electrolyte layer of step (f) with the catalytic anode formed at step (h), preferably by sealing with one or more sealants, so as to obtain a proton-conducting catalytic membrane as defined above.

In other words, according to the fifth aspect, the disclosure relates to a method for making a proton-conducting catalytic membrane as defined above, remarkable in that said method comprises the following steps:
a) providing a mixture of one or more electrolytes and one or more second metals to form a solution of mixed oxides;
b) pressing uniaxially the mixture provided at step (a) to form a porous cathode;
c) optionally, calcining said porous cathode
d) providing one or more electrolytes;
e) screen-printing said one or more electrolytes on the porous cathode or on the calcined porous cathode if step (c) is carried out, to obtain a porous cathode with an electrolyte layer;
f) sintering said porous cathode with an electrolyte layer;
g) providing an anode made of one or more first metals and/or of one or more spinels;
h) depositing at least one dehydrogenation catalyst on said electroconductive layer provided at step (g), so as to form a catalytic anode;
i) assembling together the sintered porous cathode with an electrolyte layer of step (f) with the catalytic anode formed at step (h), so as to obtain a proton-conducting catalytic membrane as defined above.

One or more of the following features advantageously further define the fourth and/or the fifth aspect:
Before step (a), an activation step is carried out under activation conditions on the oxidized form of the one or more second metals. For example, the activation conditions comprise providing a reduction atmosphere comprising preferably between 15 vol. % and 50 vol. % of hydrogen and between 85 vol. % and 50 vol. % of an inert gas based on the total volume of the reduction atmosphere. For example, the inert gas is Ar, He, N₂ or a mixture thereof, preferably Ar. For example, the activation conditions comprise a temperature ranging between 600° C. and 800° C., preferably between 650° C. and 750° C. For example, the activation conditions comprise a reduction time ranging between 5 hours and 15 hours, preferably between 7 hours and 13 hours. With preference, a calcination step is carried out before said activation step, preferably at a temperature ranging between 600° C. and 800° C., more preferably between 650° C. and 750° C. For example, the calcination step is carried out under an inert gas atmosphere, such as under Ar, He, N₂ or a mixture thereof, preferably under Ar.
The one or more electrolytes provided at step (a) is a solid solution of at least two perovskite materials.
The mixture of step (a) further comprises one or more polar aprotic solvents, such as one or more of acetone, dimethyl formamide, dimethyl sulfoxide, or a mixture thereof, preferably acetone.
The mixture provided at step (a) further comprises one or more polymers selected from polyvinyl alcohol (PVA) or poly(methyl methacrylate) (PMMA), preferably polyvinyl alcohol (PVA). For example, the mixture provided at step (a) comprises said one or more polymers and mixed oxides at a ratio ranging between 1/0.050 and 1/0.100, preferably between 1/0.060 and 1/0.090, more preferably between 1/0.070 and 1/0.080.
Step (a) is performed by grinding the mixture of one or more electrolytes and one or more second metals, preferably for at least 12 hours, more preferably for a time ranging between 18 hours and 36 hours, even more preferably ranging between 20 hours and 32 hours.
After step (a) and before step (b), a drying step is performed. For example, the drying step is performed at a temperature ranging between 40° C. and 80° C., preferably between 50° C. and 70° C.
The one or more first metals are selected from Cu, Fe, Cr, Ag, Ni, Mo, Pt, Pd, Au, Mn or any mixtures thereof, more preferably from Cu, Fe, Cr, Ag, Ni, Mo, Pt, Pd or any mixtures thereof, even more preferably from Cu, Fe, Cr, Ag, Ni, Mo or any mixtures thereof, most preferably from Cu, Fe, Cr, Ag, Ni or any mixtures thereof, such as Cu, Fe, Cr, Ni or any mixtures thereof, or such as Cu, Fe, Cr or any mixtures thereof, or such as Cu and/or Ag.
The one or more second metals are selected from Ni, Fe, Co, Pd, Pt, or any mixtures thereof, more preferably Ni.
The one or more first metals and/or the one or more spinels are doped with one or more dopants, preferably selected from Cu, Li, Cr or a mixture thereof.
Step (b) is the step of pressing uniaxially at a force ranging between 30 kN and 50 kN, preferably between 35 kN and 45 kN.
When step (c) is carried out, said step (c) is performed at a temperature ranging between 600° C. and 800° C., preferably between 650° C. and 750° C.
When step (c) is carried out, step (c) is performed during a time ranging between 5 hours and 15 hours, preferably between 7 hours and 13 hours.
The one or more electrolytes provided at step (a) and/or at step (d) are or comprise one or more perovskite materials with one or more cations selected from Ba, Ce, Zr, Y, La, Sr, Mn, Fe, Eu, Tb, Ni, Yb, Pr, Sm, Zn, Gd, Er, Co, Nd, or any mixtures thereof; with preference, said one or more cations are selected from Ba, Ce, Zr, Y, Ni or any mixture thereof.
The one or more electrolytes provided at step (a) and/or at step (d) are or comprise one or more perovskite materials with an electrical conductivity ranging between 10⁻⁴ S/cm and 10⁻³ S/cm as determined by electrochemical impedance spectroscopy measurements performed at 600° C. under an atmosphere comprising Ar and H₂ in a ratio 75/25.
The one or more electrolytes provided at step (d) are the same as the one or more electrolytes provided in the mixture with one or more second metals.
Step (f) is performed at a temperature ranging between 1300° C. and 1700° C., preferably between 1350° C. and 1650° C., more preferably between 1400° C. and 1600° C.
Step (f) is performed for a time of at least 5 hours, preferably during 10 hours and 15 hours.
Before step (h) a step of forming one or more channels within the anode provided at step (g) is carried out. With preference, said step of forming one or more channels is performed by electroforming or electroplating the anode provided at step (g). For example, said step of forming one or more channels is performed by chemical vapour deposition (CVD), physical vapour deposition (PVD), thermal spraying, microfabrication by etching, photolithography, tri-dimensional printing (such as fused deposition modelling, stereolithography or selective laser sintering), machining (such as milling, drilling or stamping), or any combination thereof.
Before step (i), a step of covering the electrochemical cell of step (f) with a layer of the same one or more first metals as those making the anode provided at step (g) is carried out.
The step (i) is performed by sealing together the electrochemical cell of step (f) with the catalytic anode formed at step (h) with one or more sealants. For example, the one or more sealants are one or more sealing tapes and/or one or more sealing pastes, more preferably one or more sealing tapes.

For example, the one or more dehydrogenation reactors are prepared according to the following method which comprises the following steps:
a) providing a reactor, preferably a reactor with a planar geometry;
b) inserting the proton-conducting catalytic membrane as defined above within said reactor and/or as produced according to the method of the fifth aspect.

### DESCRIPTION OF THE FIGURES

FIG. 1: Scheme of the installation according to the preferred embodiment of the present disclosure to conduct a polypropylene manufacturing process per the present disclosure.

FIG. 2: Sheme of the installation according to another embodiment of the present disclosure to conduct a polypropylene manufacturing process per the present disclosure.

FIG. 3: Scheme of the three layers forming the proton-conducting catalytic membrane of the present disclosure.

FIG. 4: Scanning electron microscopy image of the proton-conducting catalytic membrane of the present disclosure.

FIG. 5: Scheme of a propane dehydrogenation (PDH) process carried out with the proton-conducting catalytic membrane of the present disclosure.

FIG. 6: Scheme of a rectangular channel arranged in the electroconductive layer (i.e., the anode) of a dehydrogenation reactor of the present disclosure.

FIG. 7: Scheme of a flared depth channel arranged in the electroconductive layer (i.e., the anode) of a dehydrogenation reactor of the present disclosure, the flared depth channel having a surface area of the outlet larger than the surface area of the inlet.

FIG. 8: Representation of the mechanism when a co-ionic catalytic membrane is used.

FIG. 9: Arrangement of 4 proton-conducting catalytic membranes according to the present disclosure in a stacking forming a dehydrogenation reaction with 4 membranes working in parallel.

FIG. 10: Arrangement of 20 proton-conducting catalytic membranes according to the present disclosure in a stacking forming a dehydrogenation reaction with 20 membranes working in parallel.

FIG. 11: Electrochemical Impedance Spectroscopy (EIS) measurements of the electrolyte used in the present disclosure. Z′ and Z″ are respectively the real and the imaginary part of the impedance, each part corresponding to the two phases of the electrical impedance. The real part corresponds to the resistance R while the imaginary part corresponds to the reactance X.

FIG. 12: Zoom-in of FIG. 11 at the zone of the intersect of the curves with the abscissa.

FIG. 13: Conductivity measurement of the electrolytes used in the present disclosure. The electrolytes 1A and 1B have been pre-calcined and then sintered at 700° C. while the electrolyte 2A has been sintered at 700° C.

FIG. 14: Evolution of the electrical potential in function of the area specific resistance in the proton-conducting catalytic membrane according to the disclosure

FIG. 15: Conversion of propane into propene in function of the H₂ extraction ratio with respect to the temperature.

FIG. 16: Conversion of propane into propene in function of the H₂ extraction ratio with respect to the pressure.

FIG. 17: Propane conversion and propylene selectivity at a space velocity of 150 Nml/h/g as a function of the temperature and the hydrogen extraction ratio.

FIG. 18: Propane conversion and propylene selectivity at a space velocity of 750 Nml/h/g as a function of the temperature and the hydrogen extraction ratio.

FIG. 19: Simulation by computational fluid dynamics (CFD) of the coke formation in function of the temperature within the proton-conducting catalytic membranes of the present disclosure.

FIG. 20: Representation of a flared depth channel in accordance with the present disclosure.

FIG. 21: Evolution of the amount of coke in function of the reactor length in the absence of steam in the flared depth channel of FIG. 20.

FIG. 22: Evolution of the amount of coke in function of the reactor length when water is co-fed with the propane and when the reactor employs a proton-conducting catalytic membrane in accordance with the disclosure in the flared depth channel of FIG. 20.

FIG. 23: Evolution of the amount of coke in function of the reactor length when water is co-fed with the propane and when the reactor employs a co-ionic catalytic membrane in accordance with the disclosure in the flared depth channel of FIG. 20.

FIG. 24: Propylene yield as a function of the time on stream (TOS) comprising water at a molar fraction of 2.7%.

FIG. 25: Propylene yield as a function of the time on stream (TOS) comprising water at a molar fraction of 5.4%.

FIG. 26: Propylene yield as a function of the time on stream (TOS) comprising water at a molar fraction of 7.9%.

FIG. 27: Proton-conducting catalytic membrane in which the anode is made of copper, the membrane being incorporated within a steel housing.

FIG. 28: Proton-conducting catalytic membrane in which the anode is made of stainless steel.

FIG. 29: Photograph of the proton-conducting catalytic membrane in which the anode is made of stainless steel.

### DETAILED DESCRIPTION

For the disclosure, the following definitions are given:

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The recitation of numerical ranges by endpoints includes all integer numbers and, where appropriate, fractions subsumed within that range (e.g., 1 to 5 can include 1, 2, 3, 4, 5 when referring to, for example, a number of elements, and can also include 1.5, 2, 2.75 and 3.80, when referring to, for example, measurements). The recitation of endpoints also includes the recited endpoint values themselves (e.g., from 1.0 to 5.0 includes both 1.0 and 5.0). Any numerical range recited herein is intended to include all sub-ranges subsumed therein.

Zeolite codes (e.g., CHA . . . ) are defined according to the “Atlas of Zeolite Framework Types”, 6th revised edition, 2007, Elsevier, to which the present application also makes reference.

As used herein, the term “C# hydrocarbons”, wherein “#” is a positive integer, is meant to describe all hydrocarbons having # carbon atoms. C# hydrocarbons are sometimes indicated as just C#.

The symbol “=” in the term “C#=hydrocarbon” indicates that the hydrocarbon concerned is an olefin or an alkene, and the notation “=” symbolises the carbon-carbon double bond. For instance, “C6=” stands for “C6 olefin”, or for “olefins comprising 6 carbon atoms”.

The term “steam” is used to refer to water in the gas phase, which is formed when water boils.

The term “transition metal” refers to an element whose atom has a partially filled d sub-shell, or which can give rise to cations with an incomplete d sub-shell (IUPAC definition). According to this definition, the transition metals are Sc, Ti, V, Cr, Mn, Fe, Co, Ni, Cu, Zn, Y, Zr, Nb, Mo, Tc, Ru, Rh, Pd, Ag, Cd, La, Hf, Ta, W, Re, Os, Ir, Pt, Au, Hg, Ac, Rf, Db, Sg, Bh, Hs, Mt, Ds, Rg, and Cn.

The metals Ga, In, Sn, TI, Pb and Bi are considered “post-transition” metals.

The metals Au, Ag, Ru, Rh, Pd, Os, Ir and Pt show outstanding oxidation resistance and are considered “noble” metals. Other metals can be considered “non-noble” metals.

The term “alkali metal” refers to an element classified as an element from group 1 of the periodic table of elements (or group IA), excluding hydrogen. According to this definition, the alkali metals are Li, Na, K, Rb, Cs and Fr.

The term “alkaline earth metal” refers to an element classified as an element from group 2 of the periodic table of elements (or group IIA). According to this definition, the alkaline earth metals are Be, Mg, Ca, Sr, Ba and Ra.

The term “rare earth elements” refer to the fifteen lanthanides, as well as scandium and yttrium. The 17 rare-earth elements are cerium (Ce), dysprosium (Dy), erbium (Er), europium (Eu), gadolinium (Gd), holmium (Ho), lanthanum (La), lutetium (Lu), neodymium (Nd), praseodymium (Pr), promethium (Pm), samarium (Sm), scandium (Sc), terbium (Tb), thulium (Tm), ytterbium (Yb), and yttrium (Y).

The space velocity (Nml/h/g) is measured in term of the volumetric flow rate (Nml/h) of the reactant at 0° C. and 1.01 bar per gram of catalyst (g⁻¹). The volumetric flow rate of a fluid is expressed in Nml/h (“N” stands for “Normalized”), which corresponds to 1 cm³_{NTP}/h.

The feed flow (T/h) is measured by a flowmeter and corresponds to the amount of ton per hour that is flowing.

The particular features, structures, characteristics or embodiments may be combined in any suitable manner, as would be apparent to a person skilled in the art from this disclosure, in one or more embodiments.

The present disclosure relates to a propane dehydrogenation (PDH) process, said process is remarkable in that it comprises the following steps
a) providing a stream comprising at least propane;
b) providing at least one proton-conducting catalytic membrane, each proton-conducting catalytic membrane comprising an anode, an electrolyte layer disposed on top of the anode and a porous cathode disposed on top of the electrolyte layer, wherein the anode comprises one or more channels, each of said channels comprising at least one dehydrogenation catalyst; wherein said one or more channels are flared depth channels or rectangular channels, and whrein said one or more channels are made in copper or steel;
c) feeding within the anode of said one or more proton-conducting catalytic membranes under propane dehydrogenation conditions the stream provided at step (a);
d) recovering a first effluent comprising at least propylene;
e) optionally, recovering a second effluent comprising hydrogen.

In particular, the present disclosure relates to a propane dehydrogenation (PDH) process, said process is remarkable in that it comprises the following steps
a) providing a stream comprising at least propane;
b) providing at least one proton-conducting catalytic membrane, each proton-conducting catalytic membrane comprising an anode, an electrolyte layer disposed on top of the anode and a porous cathode disposed on top of the electrolyte layer, wherein the anode comprises one or more channels, each of said channels comprising at least one dehydrogenation catalyst; wherein said one or more channels are flared depth channels, and whrein said one or more channels are made in copper or steel;
c) feeding within the anode of said one or more proton-conducting catalytic membranes under propane dehydrogenation conditions the stream provided at step (a);
d) recovering a first effluent comprising at least propylene;
e) optionally, recovering a second effluent comprising hydrogen.

For example, the present disclosure relates to a propane dehydrogenation (PDH) process, said process is remarkable in that it comprises the following steps
a) providing a stream comprising at least propane;
b) providing at least one proton-conducting catalytic membrane, each proton-conducting catalytic membrane comprising an anode, an electrolyte layer disposed on top of the anode and a porous cathode disposed on top of the electrolyte layer, wherein the anode comprises one or more channels, each of said channels comprising at least one dehydrogenation catalyst; wherein said one or more channels are flared depth channels or rectangular channels, preferably flared depth channels, and whrein said one or more channels are made in copper or steel;
c) feeding within the anode of said one or more proton-conducting catalytic membranes under propane dehydrogenation conditions the stream provided at step (a);
d) recovering a first effluent comprising at least propylene;
e) optionally, recovering a second effluent comprising hydrogen.
   wherein the anode is devoid of pores, and wherein the electrolyte layer comprises one or more perovskite materials with one or more cations selected from Ba, Ce, Zr, Y, La, Sr, Mn, Fe, Eu, Tb, Ni, Yb, Pr, Sm, Zn, Gd, Er, Co, Nd, or any mixtures thereof.

Advantageously, the stream provided at step (a) further comprises steam, preferably in an amount ranging between 1 vol. % and 15 vol. % based on the total volume of the steam provided at step (a); more preferably ranging between 2 vol. % and 10 vol. %. For example, the amount of steam in the stream provided at step (a) is of at least 2.5 vol. % based on the total volume of the stream provided at step (a), preferably of at least 5.0 vol. %, more preferably 7.5 vol. %. For example, the process further comprises the step of providing steam into the stream provided at step (a). Said step of providing steam is performed continuously or at intervals (i.e., pulsed), preferably at intervals since this allows to remove coke that can be formed.

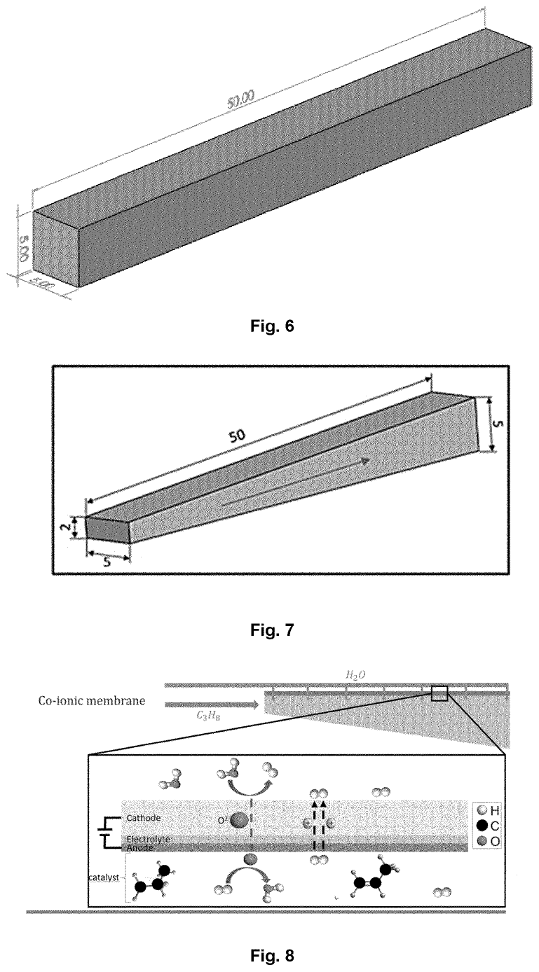

Advantageously, the proton-conducting catalytic membrane can be a co-ionic catalytic membrane. FIG. 6 displays a representation of the mechanism involving a co-ionic catalytic membrane. When a co-ionic membrane is used, water is co-fed with the feedstream (such as the feedstream of propane). The water is injected through the porous cathode of catalytic membrane, so that the water is split into hydrogen and oxygen. Then the oxygen anions can cross the membrane, so as to react with part of the hydrogen that is formed at the anode side, namely within the anode of the catalytic membrane. Such co-ionic catalytic membrane conducts therefore protons versus electrons and oxygen anions. Typical materials suitable for the co-ionic catalytic membranes are perovskites ABO₃, such as oxides of Co, oxides of CoFe, oxides of Zr, oxides of BaZr, oxides of CaZr doped with one or more of Sr, Ba, Co, Cu, Fe, Cr, La, Ni, Ca.

With preference, the control of the quantity of coke that is formed can also occur when water is co-fed with the feedstream (such as the feedstream of propane), and without the co-ionic catalytic membrane, but only in presence of the proton-conducting catalytic membrane.

The present disclosure also relates to a polypropylene manufacturing process, said process comprising the following steps:
a) providing a first propylene stream, said first propylene stream optionally comprising one or more solvents;
b) polymerizing the first propylene stream under polymerization conditions to form an effluent comprising polypropylene and at least propylene and propane and optionally the one or more solvents if any;
c) separating the effluent to generate a first stream comprising polypropylene and a second stream comprising at least propylene and propane, and optionally a third stream comprising the one or more solvents if any;
d) optionally, recycling the third stream if any;
f) splitting the second stream to produce an overhead comprising propylene and a purge stream, said purge stream being a propane-rich stream further comprising propylene;
g) optionally, recycling the overhead into the first propylene stream provided at step (a);
wherein the process is remarkable in that it further comprises a first dehydrogenating step (h) and/or a second dehydrogenating step (e); wherein said first dehydrogenating step (h) is the step of dehydrogenating the purge stream produced at step (f) to enrich in propylene the purge stream; wherein said second dehydrogenating step (e) is carried out after the separation step (c) and before the spliiting step (f) and is the step of dehydrogenating the second stream generated at step (c) to enrich in propylene the second stream; and wherein said first and second dehydrogenating steps are each a propane dehydrogation (PDH) step as defined in accordance with the first aspect.

With preference, said process comprises a first dehydrogenating step (h).

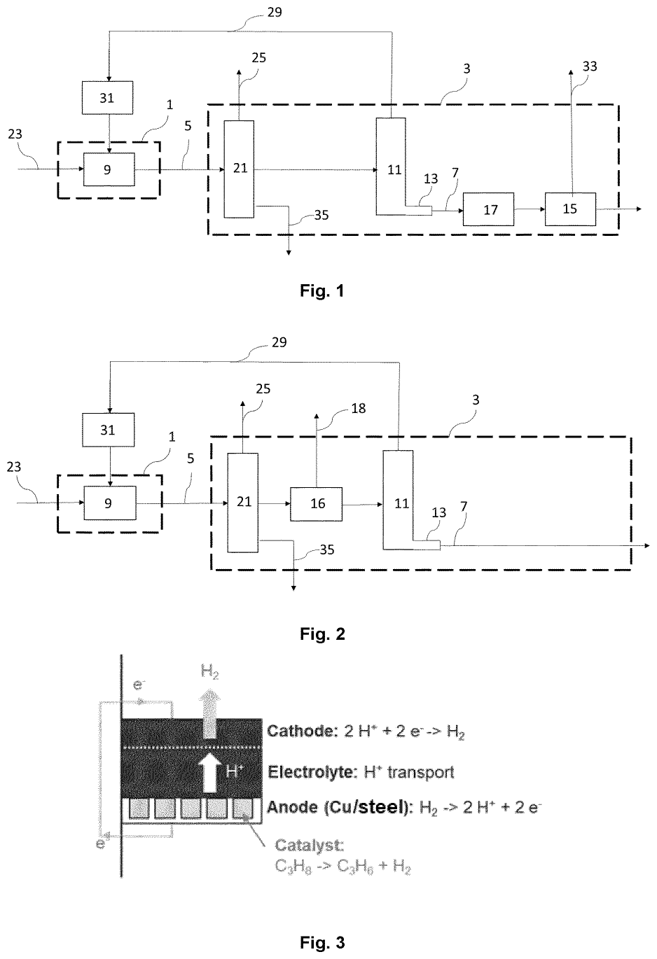

Said polypropylene manufacturing process will be described with the preferred installation into which said process will be carried out. Such preferred installation can be for example represented in FIG. 1. Other installation, such as the one represented in FIG. 2 can also be considered.

The installation comprises a polymerization zone 1 and a separation zone 3 which is downstream of the polymerization zone 1.

The polymerization zone 1 comprises a polymerization reactor 9. For example, there is an input line 23 for providing a first propylene stream as required by step (a). For example, polymer-grade propylene with a propylene content of at least 99 wt. % based on the total weight of the polymer-grade propylene, preferentially at least 99.5 wt. % is the first propylene stream. One or more catalysts, such as Ziegler-Natta catalyst or metallocene catalyst, typically titanium chlorides, one or more stabilizers such as H₂, one or more inhibitors such as H₂, and/or one or more solvents such as one or more organic solvents, and/or one or more diluents such as a C4, C5 or C6 alkanes, may be introduced into the polymerization reactor 9 as required, depending upon the specific polymerization technique being used. One or more polymerization reactors 9 can be involved in the process, with the individual reactors carrying out the same or different unit operations. The product manufactured may be any type of propylene polymer, including, but not limited to, homopolymers, such as a medium- or high-impact homopolymers; substituted, including halogenated, homopolymers; copolymers, such as random and block copolymers of ethylene and propylene; and terpolymers. For example, the polymerization step (b) is carried out in the liquid phase, resulting in the formation of polypropylene particles in suspension in the solvent, or in a gas phase, resulting in the formation of polypropylene powder entrained with the propylene gas.

It is noted that the reactor operating conditions and functioning are not critical to the disclosure and can vary from plant to plant.

As shown in FIGS. 1 and 2, the separation zone 3 comprises at least a polymer separation apparatus 21 and a splitter 11 with a bottom outlet 13, the polymer separation apparatus 21 being upstream to the splitter 11. There is a first line 5 between the polymerization reactor 9 and the polymer separation apparatus 21. The first line 5 is useful for conveying the products formed during the polymerizing step (b), namely an effluent comprising polypropylene and at least propylene (i.e., the unreacted propylene) and propane, and optionally the one or more solvents if any to the polymer separation apparatus 21, wherein the separating step (c) of the process is carried out.

For example, the polymer separation apparatus 21 can be one or more phase separation vessels (when the polymerizing step (b) is carried out in a liquid phase) or one or more cyclone separators (when the polymerizing step (b) is carried out a gas phase). The separation step (c) allows for the generation of a first stream of polypropylene that can be recovered through the polypropylene exit line 25, a second stream comprising at least propylene and propane and optionally a third stream comprising the one or more solvents if any. For example, the second stream comprising at least propylene and propane comprises after step (c) and/or before step (e) between 25 vol. % and 90 wt. % of propylene based on the total weight of the second stream, or between 30 vol. % and 85 wt. %, or between 35 vol. % and 80 wt. %.

An optional step (d) can be carried out to recycle the third stream comprising the one or more solvents. This is done via the recycling line 35. For example, the recycling line 35 can be directed to the polymerization zone 1 (not shown) to be mixed with the first propylene stream provided at step (a) and/or to downstream processes.

The installation is remarkable in that it comprises one or more first dehydrogenation reactors 15 arranged downstream of the bottom outlet 13 of the splitter and/or one or more second dehydrogenation reactors 16 arranged upstream of the splitter 11; each first and second dehydrogenation reactor (15, 16) comprising at least one proton-conducting catalytic membrane, each proton-conducting catalytic membrane comprising an anode, an electrolyte layer disposed on top of the anode and a porous cathode disposed on top of the electrolyte layer, and wherein the anode comprises one or more channels, each of said channels comprising at least one dehydrogenation catalyst, wherein said one or more channels are flared depth channels or rectangular channels, and wherein said one or more channels are made in copper or steel.

In a preferred embodiment, the installation comprises at least one first dehydrogenation reactor 15 that is arranged downstream of the bottom outlet 13 of the splitter, as depicted in FIG. 1. This allows the purge stream to be enriched with propylene.

In another embodiment, that can be complimentary to the above preferred embodiment, the installation can also comprise at least one second dehydrogenation reactor 16 arranged upstream to the splitter 11, for example between the polymer separation apparatus 21 and the splitter. This dehydrogenation reactor 16, as depicted in FIG. 2, is used to convert the propane of the second stream into propylene. The second stream comprising at least propylene and propane is thus enriched in propylene.

The one or more first and/or second dehydrogenation reactors (15, 16) of the installation can also be used to extract the hydrogen generated during the dehydrogenation reaction. The hydrogen can then be recovered in an optional step through the first hydrogen effluent line 33 (FIG. 1) and/or through the second hydrogen effluent line 18 (FIG. 2).

Advantageousy, the second stream comprises after the second dehydrogenating step (e) at least 1.5 times more propylene than before said second dehydrogenating step (e), more preferably at least 1.7 times, even more preferably at least 2.0 times. Indeed, at steady state and at steady conditions, and after catalytic bed wetting, the amount of propylene will stay identical since the propane is continuously flowing and will be continuously transformed into propylene. It can be considered that after 5 minutes of reaction during said second dehydrogenating step (e), or after 10 minutes, or after 15 minutes, or after 20 minutes, the second stream will comprise at least 1.5 times more propylene than before said second dehydrognating step (e), more preferably at least 1.7 times, even more preferably at least 2.0 times. For example, the second stream comprises after step (e) at least 50 vol. % of propylene based on the total volume of the second stream, preferably at least 60 vol. %, more preferably at least 70 vol. %, even more preferably at least 80 vol. %, most preferably at least 90 vol. %, even most preferably at least 95 vol. %.

Advantageously, the second stream further comprises after the second dehydrogenating step (e) propane, ethylene, ethane, methane, or a mixture thereof. For example, the second stream further comprises, after step (e) and in a total amount inferior to 30 vol. % of the second stream based on the total volume of said second stream, propane, ethylene, ethane, methane, or a mixture thereof; preferably inferior to 25 vol. %, more preferably inferior to 20 vol. %, even more preferably inferior to 15 vol. %, most preferably inferior to 10 vol. %, even most preferably inferior to 5 vol. %, or inferior to 2.5 vol. %, or inferior to 1 vol. %.

Advantageously, the purge stream comprises after the first dehydrogenating step (h) at least 1.5 times more propylene than before said first dehydrogenating step (h), more preferably at least 1.7 times, even more preferably at least 2.0 times. Indeed, at steady state and at steady conditions, and after catalytic bed wetting, the amount of propylene will stay identical since the propane is continuously flowing and will be continuously transformed into propylene. It can be considered that after 5 minutes of reaction during said first dehydrogenating step (h), or after 10 minutes, or after 15 minutes, or after 20 minutes, the second stream will comprise at least 1.5 times more propylene than before said first dehydrognating step (h), more preferably at least 1.7 times, even more preferably at least 2.0 times.

The second stream, that can pass through the one or more second dehydrogenation reactors 16 arranged upstream to the splitter 11, is then directed to the splitter 11. For example, the splitter 11 can be a distillation column. The splitter 11 produces an overhead comprising propylene that can be recovered and that can, optionally in step (g), be recycled via the recycling line 29 to the polymerization reactor 9 (as shown in FIG. 1) and/or to the input line 23. For example, the splitter 11 can operate at a bottom temperature ranging between 30° C. and 70° C., preferably between 35° C. and 65° C., more preferably between 40° C. and 60° C.; and/or at a pressure ranging between 0.5 MPa and 2.0 MPa, preferably between 1 MPa and 1.9 MPa, more preferably between 1.2 MPa and 1.8 MPa.

An optional dryer system 31 can be arranged on the recycling line 29, upstream of the polymerization reactor 9. For example, the dryer system 31 is a desiccant. For example, the desiccant can be a molecular sieve, such as one or more zeolite from the LTA family. Among the LTA family, zeolites from LTA-3A, LTA-4A and/or LTA-5A can be selected.

In order to protect the one or more proton-conducting catalytic membranes, and also the one or more co-ionic membranes if any, of each first and/or second dehydrogenation reactor (15, 16), a scrubber 17 (only visible on FIG. 1) is preferably placed upstream of each first and/or second dehydrogenation reactor (15, 16), more preferably directly upstream. This stage is useful to remove contaminants, such as any organoaluminium compounds coming from the polymerization reactor 9.

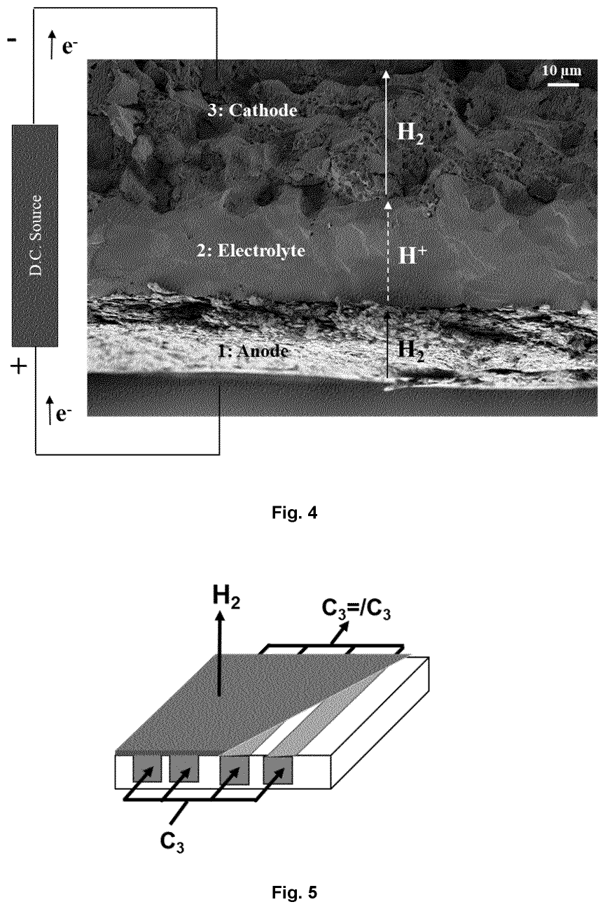

The one or more first and/or second dehydrogenation reactors (15, 16) used in the installation of the present disclosure each comprise a proton-conduction catalytic membrane, with an anode, an electrolyte layer disposed on top of the andoe and a porous cathode disposed on top of the electrolyte layer, and wherein the anode comprises one or more channels, each of said channels comprising at least one dehydrogenation catalyst, wherein said one or more channels are flared depth channels or rectangular channels, and wherein said one or more channels are made in copper or steel. The proton-conducting catalytic membrane of the present disclosure acts as an extractive membrane that removes the hydrogen that is produced during a dehydrogenation process. It is schematized in FIGS. 3 and 5 and imaged by scanning electron microscopy in FIG. 4.

During the polypropylene manufacturing process, the second stream comprising at least propylene and propane and/or the purge stream is passed within the anode of the one or more proton-conducting catalytic membranes to be dehydrogenated.

For example, the propane dehydrogenation conditions of step (e), and/or step (h), comprise one or more of the following:
providing an electrical current between the anode and the porous cathode of the one or more proton-conducting catalytic membranes. With preference, said electrical current has a density of at least 0.10 A/cm² as measured by ampere meter/power supply instrument and divided by membrane surface or calculated from conductivity measurements (from electrochemical impedance spectroscopy, EIS), more preferably of at least 0.15 A/cm², even more preferably of at least 0.20 A/cm², most preferably of at least 0.25 A/cm², even most preferably of at least 0.30 A/cm², or of at least 0.35 A/cm². For example, said electrical current has a density ranging between 0.10 A/cm² and 0.75 A/cm² as measured by ampere meter/power supply instrument and divided by membrane surface or calculated from conductivity measurements (from electrochemical impedance spectroscopy, EIS), more preferably between 0.15 A/cm² and 0.70 A/cm², even more preferably between 0.20 A/cm² and 0.65 A/cm², most preferably between 0.25 A/cm² and 0.60 A/cm², or between 0.25 A/cm² and 0.55 A/cm², or between 0.25 A/cm² and 0.50 A/cm², or between 0.30 A/cm² and 0.45 A/cm². With preference, said electrical current has electric potential ranging between 0.4 V and 1.8 V as measured by voltmeter/power supply instrument, more preferably between 0.5 V and 1.5 V; even more preferably between 0.5 V and 1.0 V.
a temperature ranging between 400° C. and 700° C., preferably between 450° C. and 650° C., more preferably between 500° C. and 600° C., even more preferably between 510° C. and 590° C., most preferably between 520° C. and 580° C., even most preferably between 525° C. and 575° C., or between 530° C. and 570° C.
a pressure ranging between 0.01 MPa and 1 MPa, preferably between 0.05 MPa and 0.9 MPa, more preferably between 0.09 MPa and 0.8 MPa, or between 0.1 MPa and 0.6 MPa.
a space velocity ranging between 100 NmL/h/g and 800 NmL/h/g as measured by the flowrate from floweter divided by the catalyst mass, preferably between 200 NmL/h/g and 700 NmL/h/g, more preferably between 300 NmL/h/g and 600 NmL/h/g.

Advantageously, the propane dehydrogenation conditions of step (c) comprise a temperature ranging between 425° C. and 575° C.; preferably between 430° C. and 570° C., more preferably between 435° C. and 565° C. For example, the propane dehydrogenation conditions comprise a space velocity of at least 550 Nml/h/g, or of at least 600 Nml/h/g, or of at least 650 Nml/h/g, or of at least 700 Nml/h/g, or of at least 750 Nml/h/g. For example, the propane dehydrogenation conditions comprise a space velocity ranging between 550 Nml/h/g and 1000 Nml/h/g, or between 600 Nml/h/g and 1000 Nml/h/g, or between 650 Nml/h/g and 1000 Nml/h/g, or between 700 Nml/h/g and 1000 Nml/h/g, or between 750 Nml/h/g and 1000 Nml/h/g

For example, the second stream has during step (e) a feed flow ranging between 20 T/h and 40 T/h as measured by a flowmeter, preferably between 22 T/h and 38 T/h, or between 24 T/h and 36 T/h, or between 26 T/h and 32 T/h.

For example, the purge stream has during step (h) a feed flow ranging between 0.5 T/h and 5 T/h as measured by a flowmeter, preferably between 1 T/h and 4 T/h, or between 1.5 T/h and 3.5 T/h.

The proton-conducting catalytic membrane is made according to the following method, which comprises the following steps:
a) providing a mixture of one or more electrolytes and one or more second metals to form a solution of mixed oxides;
b) pressing uniaxially the mixture provided at step (a) to form a porous cathode, for example at a force ranging between 30 kN and 50 kN, preferably between 35 kN and 45 kN;
c) optionally, calcining said porous cathode;
d) providing one or more electrolytes;
e) screen-printing said one or more electrolytes on the porous cathode or on the calcined porous cathode if step (c) is carried out, to obtain an electrochemical cell with an electrolyte layer;
f) sintering said electrochemical cell;
g) providing an electroconductive layer made of one or more first metals and/or of one or more spinels;
h) depositing at least one dehydrogenation catalyst on said electroconductive layer provided at step (g), so as to form a catalytic electroconductive layer;
i) assembling together the electrochemical cell of step (f) with the catalytic electroconductive layer formed at step (h), preferably by sealing with one or more sealants, so as to obtain a proton-conducting catalytic membrane in accordance with the present disclosure.

The screen-printing technology, used at step (e), is a printing technique where a mesh, for example a 21 mesh, is used to transfer an ink onto a substrate. In this case, the substrate is a porous layer which corresponds to the cathode and the ink is made essentially of the electrolyte.

The steps (a) to (f) are the steps for preparing a sintered electrochemical cell with an electrolyte layer, which corresponds to the cathode of a proton-conducting membrane or of a proton-conducting catalytic membrane.

Before step (a), an activation step can be carried out under activation conditions on the oxidized form of the one or more second metals. For example, the activation conditions comprise providing a reduction atmosphere comprising preferably between 15 vol. % and 50 vol. % of hydrogen and between 85 vol. % and 50 vol. % of an inert gas based on the total volume of the reduction atmosphere. For example, the inert gas is Ar, He, N₂ or a mixture thereof, preferably Ar. For example, the activation conditions comprise a temperature ranging between 600° C. and 800° C., preferably between 650° C. and 750° C. For example, the activation conditions comprise a reduction time ranging between 5 hours and 15 hours, preferably between 7 hours and 13 hours. With preference, a calcination step is carried out before said activation step, preferably at a temperature ranging between 600° C. and 800° C., more preferably between 650° C. and 750° C. For example, the calcination step is carried out under an inert gas atmosphere, such as under Ar, He, N₂ or a mixture thereof, preferably under Ar.

The one or more electrolytes provided at step (a) can be a solid solution of at least two perovskite materials. The mixture of step (a) can further comprise one or more polar aprotic solvents, such as one or more of acetone, dimethyl formamide, dimethyl sulfoxide, or a mixture thereof, preferably acetone.

The mixture provided at step (a) can further comprise, to favour the pressing, one or more polymers selected from polyvinyl alcohol (PVA) or poly(methyl methacrylate) (PMMA), preferably polyvinyl alcohol (PVA). For example, the mixture provided at step (a) comprises said one or more polymers and mixed oxides at a ratio ranging between 1/0.050 and 1/0.100, preferably between 1/0.060 and 1/0.090, more preferably between 1/0.070 and 1/0.080.

Step (a) can be performed by grinding the mixture of one or more electrolytes and one or more second metals, preferably for at least 12 hours, more preferably for a time ranging between 18 hours and 36 hours, even more preferably ranging between 20 hours and 32 hours.

After step (a) and before step (b), a drying step can be performed. For example, the drying step is performed at a temperature ranging between 40° C. and 80° C., preferably between 50° C. and 70° C.

The one or more first metals are advantageously selected from Cu, Ag, Ni, Pt, Pd, Au, Mn, or any mixtures thereof, more preferably Cu and/or Ag. The one or more first metals and/or the one or more spinels can be doped with one or more dopants, preferably selected from Cu, Li, Cr or a mixture thereof.

The one or more second metals are advantageously selected from Ni, Fe, Co, Pd, Pt, or any mixtures thereof, more preferably Ni.

When step (c) is carried out, said step (c) can be performed at a temperature ranging between 600° C. and 800° C., preferably between 650° C. and 750° C.; and/or during a time ranging between 5 hours and 15 hours, preferably between 7 hours and 13 hours.

The one or more electrolytes provided at step (a) and/or at step (d) can be or comprise one or more perovskite materials with one or more cations selected from Ba, Ce, Zr, Y, La, Sr, Mn, Fe, Eu, Tb, Ni, Yb, Pr, Sm, Zn, Gd, Er, Co, Nd, or any mixtures thereof; with preference, said one or more cations are selected from Ba, Ce, Zr, Y, Ni or any mixture thereof. In addition, the use of one or more perovskite materials with one or more cations selected from Ba, Ce, Zr, Y, La, Sr, Mn, Fe, Eu, Tb, Ni, Yb, Pr, Sm, Zn, Gd, Er, Co, Nd, or any mixtures thereof as the electrolyte layer favours the proton transport from the anode to the cathode. For example, they are or comprise one or more perovskite materials with an electrical conductivity ranging between 10⁻⁴ S/cm and 10⁻³ S/cm as determined by electrochemical impedance spectroscopy measurements performed at 600° C. under an atmosphere comprising Ar and H₂ in a ratio 75/25. Advantageously, the one or more electrolytes provided at step (d) are the same as the one or more electrolytes provided in the mixture with one or more second metals.

Step (f) can be performed at a temperature ranging between 1300° C. and 1700° C., preferably between 1350° C. and 1650° C., more preferably between 1400° C. and 1600° C.

Step (f) can be performed for a time of at least 5 hours, preferably during 10 hours and 15 hours.

The steps (g) and (i) are the steps of making an anode on the electrolyte layer of the sintered electrochemical cell, the anode comprising ideally one or more dehydrogenation catalysts provided at step (h) so that the proton-conducting membrane is a proton-conducting catalytic membrane and can be used for example in a dehydrogenation process.

Before step (h) a step of forming one or more channels within the electroconductive layer provided at step (g) can be carried out. With preference, said step of forming one or more channels is performed by electroforming or electroplating the electroconductive layer provided at step (g). For example, said step of forming one or more channels is performed by chemical vapour deposition (CVD), physical vapour deposition (PVD), thermal spraying, microfabrication by etching, photolithography, tri-dimensional printing (such as fused deposition modelling, stereolithography or selective laser sintering), machining (such as milling, drilling or stamping), or any combination thereof.

Before step (i), a step of covering the electrochemical cell of step (f) with a layer of the same one or more first metals as those making the electroconductive layer provided at step (g) can be carried out. The step (i) is advantageously performed by sealing together the electrochemical cell of step (f) with the catalytic electroconductive layer formed at step (h) with one or more sealants. For example, the one or more sealants are one or more sealing tapes and/or one or more sealing pastes, more preferably one or more sealing tapes.

### The Anode

The anode is an electroconductive layer. The anode comprises at least one dehydrogenation catalyst. The anode is preferably devoid of pores.

For example, the electroconductive layer is made of one or more first metals and/or of one or more spinels (i.e., MgAl₂O₄). This corresponds to an anode, where hydrogen is transformed into protons (H⁺), following the chemical equation (1):

H₂→2H⁺+2e⁻

With preference, the one or more first metals are selected from Cu, Fe, Cr, Ag, Ni, Mo, Pt, Pd, Au, Mn or any mixtures thereof, more preferably from Cu, Fe, Cr, Ag, Ni, Mo, Pt, Pd or any mixtures thereof, even more preferably from Cu, Fe, Cr, Ag, Ni, Mo or any mixtures thereof, most preferably from Cu, Fe, Cr, Ag, Ni or any mixtures thereof, such as Cu, Fe, Cr, Ni or any mixtures thereof, or such as Cu, Fe, Cr or any mixtures thereof, or such as Cu and/or Ag. Silver is the highest conducting metals while copper is the cheapest. The one or more first metals and/or the one or more spinels can be preferably doped with one or more dopants which can be selected from Cu, Li, Cr or a mixture thereof.

Advantageously, the anode is made of or comprises steel.

For example, the anode is made of or comprises stainless steel, more preferably ferritic stainless steel.

For example, the steel comprises between 60 wt. % and 80 wt. % of iron based on the total weight of the steel, preferably between 63 wt. % and 75 wt. % of iron.

For example, the steel comprises between 11 wt. % and 18 wt. % of chromium based on the total weight of the steel, preferably between 12 wt. % and 17 wt. %.

For example, the steel comprises between 10 wt. % and 14 wt. % of nickel based on the total weight of the steel, preferably between 11 wt. % and 13 wt. %.

For example, the steel comprises between 1 wt. % and 3 wt. % of molybdenum, such as 2 wt. % of molybdenum.

For example, the steel comprises less than 0.15 wt. % of carbon based on the total weight of the steel, preferably less than 0.12 wt. %.

For example, the steel comprises between 60 wt. % and 80 wt. % of iron, between 11 wt. % and 18 wt. % of chromium and less than 0.15 wt. % of carbon, based on the total weight of the steel.

For example, the steel comprises at least 60 wt. % of iron and between 11 wt. % and 18 wt. % of chromium based on the total weight of the steel. With preference, the steel also comprises less than 0.15 wt. % of carbon based on the total weight of steel; preferably less than 0.12 wt. %.

In particular, the steel comprises at least 60 wt. % of iron, between 11 wt. % and 18 wt. % of chromium, and less than 0.15 wt. % of carbon, based on the total weight of the steel. More particularly, the steel comprises at least 60 wt. % of iron, between 11 wt. % and 18 wt. % of chromium, less than 0.15 wt. % of carbon, between 10 wt. % and 14 wt. % of nickel and between 1 wt. % and 3 wt. % of molybdenum, based on the total weight of the steel.

Advantageously, the anode has a thickness ranging between 3 mm and 6 mm as designed by SolidWorks software, preferably between 3.5 mm and 5.5 mm. A reactor housing, made for example in Inconel or steel, can encompass the anode, such as an anode made of copper.

When the anode is made of steel, there is no need of reactor housing. Therefore, the thickness of the whole reactor is ranging between 1 cm and 2 cm. As no reactor housing is required, the reactor is lighter compared to a reactor with an anode in copper which required an Inconel or a steel housing.

For example, the anode is non-porous.

Advantageously, the one or more dehydrogenation catalysts comprise one or more metal-based catalysts, one or more metal oxide-based catalysts, one or more zeolites, one or more transition metal carbides, one or more transition metal nitrides, one or more carbon nanotubes, or any mixtures thereof. With preference, the one or more dehydrogenation catalysts comprise one or more metal-based catalysts, one or more metal oxide-based catalysts, one or more zeolites, or any mixtures thereof. For example, the one or more zeolites are selected from the group of CHA, MFI families or any mixtures thereof. For example, the one or more zeolites are doped with one or metals selected from Mo, W, Fe, V, Cr or any mixtures thereof. For example, the one or more metal-based catalysts and/or the one or more metal oxide-based catalyst comprises one or more metals selected from Pd, Pt, Sn, Mo, W, Fe, V, Cr or any mixtures thereof. The metal-based catalysts, such as the catalysts comprising one or more metals selected from Pd, Pt, Sn, Mo, W, Fe, V, Cr or any mixtures thereof, or preferably selected from Pd, Pt, Sn or any mixture thereof, are preferred as dehydrogenation catalysts. Such metal-based catalysts can be supported, for example onto Al₂O₃, SiO₂, TiO₂, CeO₂ or any mixture thereof, more preferably Al₂O₃ and/or SiO₂.

The fact that the anode comprises the one or more dehydrogenation catalysts allows for maximisation of the contact surface between the one or more dehydrogenation catalysts and the electrolyte, leading therefore to an improvement of the extraction capability of the membrane.

### The Electrolyte Layer

The electrolyte layer acts as a proton transport layer. It is noted that since the electrolyte is a medium containing ion and that is electrically conducting upon the movement of those ions, the alkanes such as the propane, or the products of the dehydrogenation reaction, namely the corresponding alkenes, such as the propene, cannot cross the electrolyte layer. Only the hydrogen under the form of proton (H+) will be able to cross the electrolyte layer and then extracted upon application of an electrical current.

For example, the electrolyte of the electrolyte layer comprises one or more perovskite materials with an electrical conductivity ranging between 10⁻⁴ S/cm and 10⁻³ S/cm as determined by electrochemical impedance spectroscopy measurements performed at 600° C. under an atmosphere comprising Ar and H₂ in a ratio 75/25.

For example, the electrolyte of the electrolyte layer comprises one or more perovskite materials having a general formula ABX₃, wherein A and B are cations with different oxidation states and X is an anion. The A-cation occupies the center of the unit cell, while the B cation and the X anions (commonly oxygen) are arranged at the corners and the edges of the unit cell, respectively.

For example, the electrolyte of the electrolyte layer comprises one or more perovskite materials with one or more cations selected from Ba, Ce, Zr, Y, La, Sr, Mn, Fe, Eu, Tb, Ni, Yb, Pr, Sm, Zn, Gd, Er, Co, Nd, or any mixtures thereof. With preference, said one or more cations are selected from Ba, Ce, Zr, Y, Ni or any mixtures thereof. More preferably, the one or more cations are selected from Ba, Ce, Zr, Y, or any mixtures thereof. For example, the electrolyte comprises at least two perovskite materials, preferably in the form of a solid solution. The use of such cations as the electrolyte layer favours the proton transport from the anode to the cathode.

For example, the electrolyte of the electrolyte layer comprises one or more perovskite materials being BaCeO₃ and BaZrO₃ in the form of a solid solution. BaCeO₃ exhibits higher proton conductivity than BaZrO₃, but can suffer chemical instability. On the other hand, BaZrO₃ presents adequate stability under different conditions but presents a significant grain boundary resistance in addition to the high sintering temperature that causes Ba evaporation and the subsequent loss of transport properties. The solid solution of both BaCeO₃ and BaZrO₃, corresponding to the electrolyte BaCe_{0.3}Zr_{0.5}Y_{0.2}O₃, can overcome the disadvantages of both materials taken independently.

Advantageously, the electrolyte layer has a thickness ranging between 20 μm and 40 μm as determined by scanning electron microscopy, preferably between 25 μm and 35 μm.

With preference, the anode, which is an electroconductive layer, comprises one or more channels arranged in parallel to each other, each of said channels comprising the one or more dehydrogenation catalysts. For example, the anode can advantageously comprise two channels, preferably 4 channels, more preferably 8 channels, or 9 channels or 10 channels. A configuration in which an anode comprises 4 channels is schematized in FIG. 5. The fact that the one or more dehydrogenation catalysts are surrounded by the electrolyte further contributes to the extractive capabilities of the proton-conducting catalytic membrane of the present disclosure. For example, each of said one or more channels has a cross-section amounting to at least 2 cm², or to at least 2.5 cm². For example, at least a part of said one or more channels are rectangular channels (see FIG. 6), flared depth channels (see FIG. 7), flared width channels, bended channels, or corrugated channels; more preferably, all of said one or more channels are rectangular channels, flared depth channels, flared width channels, bended channels, or corrugated channels; even more preferably, all of said one or more channels are rectangular channels or flared depth channels, most preferably, all of said one or more channels are flared depth channels. In particular, flared depth channels are channels with a surface area of the outlet larger than the surface area of the inlet, the inlet and the outlet being defined according to the sense of the feed flow, for example the inlet is where the compound to be dehydrogenated (e.g., the propane) is introduced into the proton-conducting catalytic membrane and the outlet is where the one or more products of the dehydrogenation reaction (e.g., propylene, cracked products (such as ethane and/or methane), coke) are exiting the proton-conducting catalytic membrane.

### The Porous Cathode

For example, the porous cathode comprises a mixture of an electrolyte and one or more second metals. This corresponds to an electrochemical cell with a porous cathode, which ensures the recombination of protons (H⁺) into hydrogen (H₂), following the chemical equation (2):

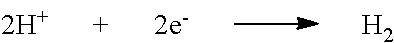

With preference, the one or more second metals are selected from Ni, Fe, Co, Pd, Pt, or any mixtures thereof, more preferably Ni. For example, the amount of the one or more second metals is ranging between 50 wt. % and 70 wt. % of the total weight of said mixture, more preferably between 55 wt. % and 65 wt. %. Thus in the case where nickel would be selected as the preferred cathodic metal, the amount of nickel in the mixture forming the porous cathode is ranging between 50 wt. % and 70 wt. % of the total weight of said mixture, more preferably between 55 wt. % and 65 wt. %.

Advantageously, the porous cathode is a porous layer with a thickness ranging between 30 μm and 50 μm as determined by scanning electron microscopy, preferably between 35 μm and 45 μm.

### The Proton-Conducting Catalytic Membrane

The proton-conducting membrane conducts protons versus electrons.

Advantageously, the proton-conducting catalytic membrane of the present disclosure has a hydrogen extraction ratio ranging between 0.20 and 0.99, or between 0.25 and 0.90at a temperature of at least 500° C. and/or at a pressure of at most 0.1 MPa; the hydrogen extraction ratio being simulated by computational fluid dynamics (CFD) and/or quantified by gas chromatography (GC) during the reaction. With preference, the proton-conducting catalytic membrane presents a hydrogen extraction ratio ranging between 0.20 and 0.99, or between 0.25 and 0.90 at a temperature of at least 550° C. and/or at a pressure of at most 0.05 MPa.

For example, the hydrogen extraction ratio can be of at least 0.2 at least 600° C., the hydrogen extraction ratio being simulated by computational fluid dynamics (CFD), or the hydrogen extraction ratio can be of at least 0.5 at least 575° C.

Advantageously, the proton-conducting catalytic membrane can be a co-ionic catalytic membrane. FIG. 8 displays a representation of the mechanism involving a co-ionic catalytic membrane. When a co-ionic membrane is used, water is co-fed with the feedstream (such as the feedstream of propane). The water is injected through the porous cathode of catalytic membrane, so that the water is split into hydrogen and oxygen. Then the oxygen anions can cross the membrane, so as to react with part of the hydrogen that is formed at the anode side. Such catalytic membrane conducts therefore protons versus electrons and oxygen anions and can therefore be referred to as a co-ionic catalytic membrane. Typical materials suitable for the co-ionic catalytic membranes are perovskites ABO₃, such as oxides of Co, oxides of CoFe, oxides of Zr, oxides of BaZr, oxides of CaZr doped with one or more of Sr, Ba, Co, Cu, Fe, Cr, La, Ni, Ca.

With preference, the control of the quantity of coke that is formed can also occur when water is co-fed with the feedstream (such as the feedstream of propane), and without the co-ionic catalytic membrane, but only in presence of the proton-conducting catalytic membrane.

### The Dehydrogenation Reactor

The present disclosure also relates to a dehydrogenation reactor comprising at least one proton-conducting catalytic membrane. With preference, the dehydrogenation reactor has a planar geometry, reducing subsequently the gas polarization inside the reactor. It is therefore possible to further improve the extracting capability of the proton-conducting catalytic membrane of the present disclosure. Such dehydrogenation reactor further minimizes coking formation and therefore preserves the activity of the one or more dehydrogenation catalysts.

A method for preparing the dehydrogenation reactor is also described. Said method of preparation comprises the following steps:
a) providing a reactor, preferably a reactor with a planar geometry;
b) inserting the proton-conducting catalytic membrane as defined above within said reactor.

For example, when said dehydrogenation reactor comprises more than one proton-conducting catalytic membrane, said dehydrogenation reactor further comprises a spacer between each proton-conducting membrane.

For example, the spacer is an electroconducting plate, preferably a steel plate, more preferably a stainless steel plate, even more preferably a ferritic stainless steel plate. For example, the steel plate is doped with one or more oxides (such as oxides of La and/or Y), one or more metals, (such as metals selected from Au, Ni, Pt, Cu, Pd, Ti, W or a mixture thereof), one or more metallic alloys (such as one or more ferritic alloys and/or one or more chromite alloys) or a mixture thereof. The dehydrogenation reactor can thus be advantageously an arrangement of several proton-conducting catalytic membranes. For example, the arrangement of several proton-conducting catalytic membranes can comprise two or more proton-conducting catalytic membranes, preferably between 4 and 20 proton-conducting catalytic membranes.

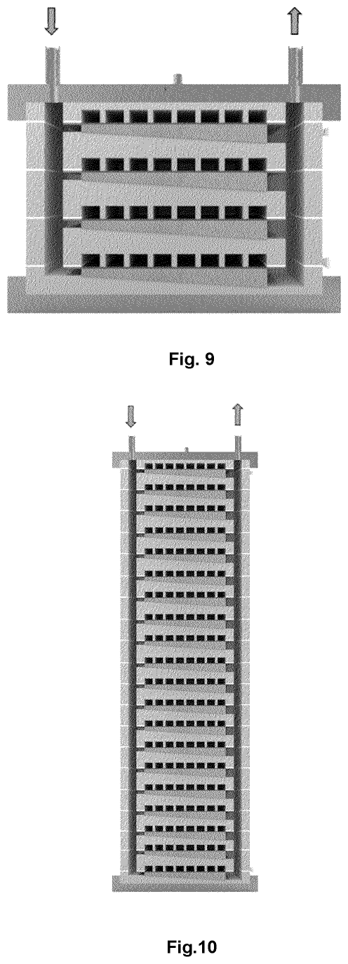

The several proton-conducting catalytic membranes can be arranged on top of each other, so as in FIG. 8 (namely a stacking of 4 membranes) or in FIG. 9 (namely a stacking of 20 membranes), or in an adjacent manner, wherein each proton-conducting catalytic membranes can be arranged next to each other on the same level. In another configuration, the several proton-conducting catalytic membranes can be arranged both on top of each other and in an adjacent manner.

Advantageously, said reactor has an inlet and an outlet, and said reactor is remarkable in that the inlet is in a first zone arranged so that a feed to said reactor via the inlet contacts the one or more dehydrogenation catalysts of the proton-conducting catalytic membrane before passing to the outlet, the outlet being for example in a second zone that is downstream of the proton-conducting catalytic membrane.

Advantageously, said reactor has an inlet and an outlet, and said reactor is remarkable in that the anode of the one or more proton-conducting catalytic membranes comprises one or more channels, each of said channels comprising the one or more dehydrogenation catalysts, and in that the inlet is in a first zone arranged so that a feed to said reactor via the inlet is conducted through the one or more channels and contacts the one or more dehydrogenation catalysts before passing to the outlet being for example in a second zone that is downstream of the proton-conducting catalytic membrane.

With preference, the inlet has surface area identical to the surface area of the outlet or the inlet has a surface area smaller than the surface area of the outlet. More preferably, the inlet has a surface area smaller than the surface area of the outlet, which is the case when the channels within the anode are flared depth channels.

For example, the surface area of the inlet can represent between 25% and 50% of the surface area of the outlet, preferably between 30% and 45%. In other words, the surface area of the outlet can be at least 2 times larger than the surface area of the inlet, more preferably at least 2.5 times, even more preferably at least 2.75 times, most preferably at least 3 times. This gradient in the geometry, wherein the lowest surface area is at the inlet side of the reactor and the bigger surface area is at the outlet side of the reactor, favours the extraction of hydrogen at the outlet side, in accordance with the circulation of the feed within the dehydrogenation reactor.

With preference, the inlet and outlet of the reactor are arranged on faces of the reactor that are opposed to each other. In other words, the inlet is in a first zone arranged so that a feed to said reactor via the inlet is conducted towards the proton-conducting catalytic membrane and the outlet is in a second zone that is downstream of the proton-conducting catalytic membrane. With preference, the first zone and the second zone each form one face of the reactor and are advantageously opposed to each other. Alternatively, the first zone and the second zone are arranged on a single face of the reactor, as shown by the stacking of FIGS. 9 and 10.

With preference, said reactor has an additional inlet and an additional outlet arranged on faces opposed to each other. This additional inlet can be used for feeding a carrier gas, such as H₂, Ar, HArMix (i.e., a mixture comprising H₂ and Ar with an amount of hydrogen comprised between 3 vol. % and 5 vol. % of the total volume of the mixture), or any mixtures thereof, to the dehydrogenation reactor.

### Test and Determination Methods

Scanning electron microscopy (SEM) analysis was carried out by using a field-emission scanning electron microscope using a Zeiss Ultra 55 fitted with a field emission gun using an accelerating voltage of 30.0 kV. All samples before the SEM characterization were covered with a conductive layer (Pt or Au).

Electrochemical Impedance Spectroscopy (EIS) measurements were performed to validate the transport properties of the developed membranes. EIS measurements were measured using a SolarTron instrument and by applying an electrical current through the membrane by means of two silver electrodes EIS allows obtaining the resistance of the electrolyte, while equation (1) allows the determination of the electrical conductivity of the material.

$$σ ( \frac{S}{cm} ) = \frac{1}{R ( Ω )} * \frac{t ( cm )}{ϕ ( {cm}^{2} )} \qquad ( 1 )$$

wherein σ is the electrical conductivity, R is the electrical resistance and φ is the area of electrode and t is the thickness of the electrode.

The hydrogen extraction ratio, which is the quantity of hydrogen extracted on the quantity of hydrogen formed, is obtained by simulations using computational fluid dynamics (CFD) technique and/or is determined by using gas chromatography (GC) technique. The hydrogen extraction ratio is calculated and/or determined on the basis of the reactant conversion, for example the propane conversion and in the case of GC, the quantity of hydrogen formed, or in the case of CFD, the theoretical quantity of hydrogen generated.

With respect to the simulations made by CFD technique, they were performed with COMSOL Multiphysics 5.6 software on SYS-6018R-MTR Super server, with an Intel Xeon CPU E5-2640 v4 processor (clock speed 2.4 GHZ, 40 CPUs) and 131 Gb RAM running Windows server edition 2016 (64-bit) as an operating system. For the gas flow, Navier-Stokes equations with the respective correction for the porous catalytic bed has been considered. Transport of species has been modelled using averaged-mixture model with propane, propylene and H₂ as species. The density of the gas mixture has been estimated considering a mixture of ideal gases, and the viscosity has been calculated using the Wilke model. The porosity and permeability are two key factors that govern the fluid flow in the porous region and the permeability for a packed bed with randomly distributed spherical particles was calculated using the Carman-Kozeny model. According to the publication of Tong Y. et al (Int. J. of Hydrogen Energy, 2022, 47, 12067-12073), electrochemical hydrogen pumps are devices based upon proton-conducting electrolytes that offer 100% selectivity to hydrogen and allow for easy control of the hydrogen separation rate by simply adjusting the applied direct current. The tortuosity was estimated considering the inverse of the square root of the porosity. The binary gas diffusion coefficient has been calculated with an empirical equation based on the Fuller kinetic gas theory, and it was corrected with the ratio of the porosity to tortuosity. The mesh performed was based on tetrahedral elements, where the element size was calibrated for fluid dynamics. The calculations were carried out using the Parallel Direct Solver (PARDISO) with parameter continuation to assure convergence. The relative tolerance of the method is 0.001.

The selectivity in propylene (C₃=) is determined according to formula (2):

$${S}_{propylene} = \frac{3 [ {C}_{3} {H}_{6} ]}{[ {CH}_{4} ] + 2 [ {C}_{2} {H}_{4} ] + 2 [ {C}_{2} {H}_{6} ] + 3 [ {C}_{3} {H}_{6} ] + 3 [ {C}_{3} {H}_{8} ]} \times 100 \qquad ( 2 )$$

wherein the numerator is the carbon adjusted molar amount of propylene and the denominator is the sum of all the carbons adjusted molar amount of all hydrocarbons in the effluent. Propylene yield was determined by multiplying the propane conversion with the propylene selectivity and divided by 100.

The thickness of the anode has been designed using SolidWorks software, which is a solid modelling computer-aided design (CAD) and computer-aided engineering (CAE) application published by Dassault Systems.

The feed flow of the second stream and/or of the purge stream has been determined using a SolarTron ISA flowmeter.

The ampere meter/power supply instrument and the voltmeter/power supply instrument used for determining respectively the electrical current density and the electrical potential is a SolarTron power supply module.

### EXAMPLES

The embodiments of the present disclosure will be better understood by looking at the different examples below.

Preparation of the Electrolyte BaCe_{0.3}Zr_{0.5}Y_{0.2}O₃ Under a Form of a Solid Solution of BaCeO₃ and BaZrO₃.
1) Grind the precursors separately for 60 hours. The employed precursors were: BaCO₃ (99.8% 1-micron powder. Alfa Aesar Ref: 14341, 1 kg, CAS: 513-77-9), YSZ (ZrO₂/Y₂O₃) (8%), CeO₂ (REacton, 99.9% (REO), 5-micron powder. Alfa Aesar Ref: 11328, 1 kg, CAS: 1306-38-3) and Y₂O₃ (99.9995%. ABCR Ref: AB102097, 100 g, CAS: 1314-36-9).
2) Mix the different precursors in adequate proportions in acetone to fulfil the stoichiometry of the desired compound and grind for 24 hours. The following amount of precursors have been weighted:
BaCO₃→17.06 g
YSZ→6.14 g
CeO₂→4.46 g
Y₂O₃→1.09 g
3) Dry by heating at 60° C. in a furnace.
4) Sieve the oxide mixture with a particle size of 200 μm.
5) The oxide mixture is uniaxially pressed at 40 kN in the shape of a disc (disc diameter: 30 mm).
6) Sinter discs at 1100° C. for 10 hours.
7) Repeat steps 4-6 twice then, grind the mixture for 24 hours.
8) The calcined oxide mixture is pressed on 20 mm discs at 30 kN.
9) Sinter the obtained discs at 1565° C. for 12 hours.
   Preparation of the cathode NiO—BaCe_{0.3}Zr_{0.5}Y_{0.2}O₃ under a form of a solid solution of BaCeO₃ and BaZrO₃

25 g of the solid solution of BaCeO₃ and BaZrO₃ is then mixed and ground for 15 hours in acetone together with 37.5 g NiO (99%-metal basis; 325 mesh powder, Alfa Aesar Ref: 12359, 250 g, CAS 1313-99-1). After a step of drying by heating at 60° C. in a furnace, the mixed oxides are mixed with polyvinyl alcohol (PVA) to favour the pressing. The mixture has a ratio of mixed oxides/PVA 1/0.075. It is then ground with Agata's mortar before being pressed uniaxially at 40 kN in the shape of a disc (disc diameter: 20 mm). Optionally, the cathode is calcined at 700° C. for 10 h.

Then, the electrolytes are coated on the cathode by using screen-printing technique using a 21-mesh to obtain a porous cathode with an electrolyte layer. The support is a pressed powder transformed into a disk. In membranes, the screen-printing procedure consists of squeezing the slurry “ink” (electrolyte) to pass through a 21-mesh screen to print it on the cathode. After drying the layer (temperature of 80° C.), it is possible to deposit extra layers. The typical thickness of the obtained layer is around 30 μm. After that, the different layers and cathode are sintered at 1565° C. temperature to obtain an efficient attachment between the cathode and the electrolyte and to afford a sintered porous cathode with an electrolyte layer.

Prior to be used, the NiO species have been reduced under hydrogen to form Nit. This activation step is carried out in a 25 vol. % H₂ atmosphere in argon at 700° C. for 10 hours, optionally with a pre-calcination at 700° C. under pure argon for 10 hours before the activation step.

### Preparation of the Proton-Conducting Membrane

Then, an anodic layer is coated on the electrolyte layer of the sintered porous cathode.

The anodic layer is an anode comprising Ag. A silver conducting paint (DYNALOY® 342) was indeed obtained from Merck (CAS 7440-22-4) and painted by hand.

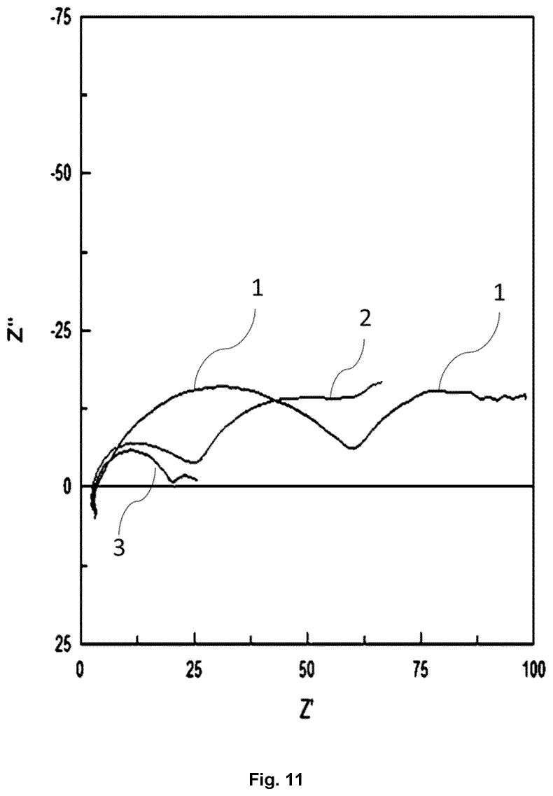

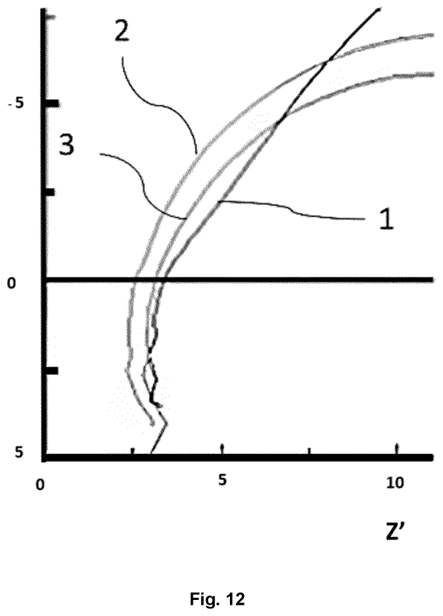

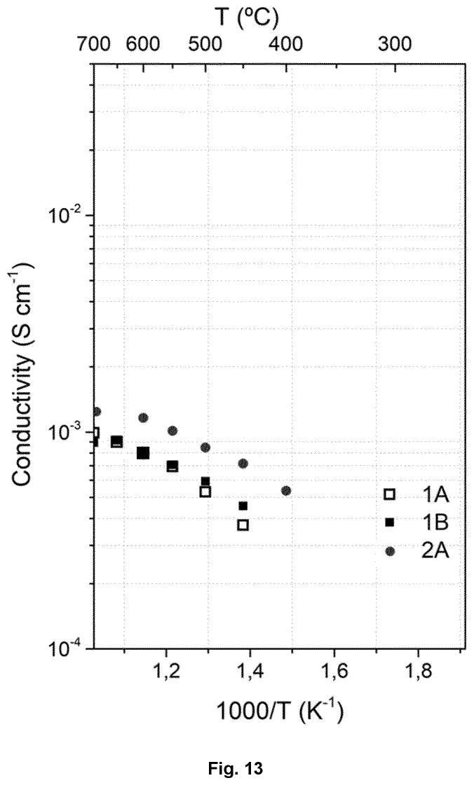

FIG. 11 shows a representative example of the electrochemical impedance spectra used to calculate the electrical conductivity. Three batches proton-conducting membrane, namely of electrochemical cells (BaCe_{0.3}Zr_{0.5}Y_{0.2}O₃ on uncalcined NiO—BaCe_{0.3}Zr_{0.5}Y_{0.2}O₃) layered with an Ag anode have been used. The curve “1” corresponds to wet conditions test (i.e., 3 vol. % of water in the H₂/Ar feed) while the curves “2” and “3” are under the same dry conditions (i.e., H₂/Ar feed without water) (reproducibility test). The tests have been made at 700° C. The intersect with the abscissa (that can be viewed on the zoom of FIG. 11 provided at FIG. 12) gives a value used for plotting FIG. 13.

FIG. 13 shows the conductivity of three batches of proton-conducting membranes, namely of electrochemical cells (1A: BaCe_{0.3}Zr_{0.5}Y_{0.2}O₃ on uncalcined NiO—BaCe_{0.3}Zr_{0.5}Y_{0.2}O₃ with an activation preceded by a calcination; 1B: BaCe_{0.3}Zr_{0.5}Y_{0.2}O₃ on uncalcined NiO—BaCe_{0.3}Zr_{0.5}Y_{0.2}O₃ with an activation without calcination; and 2A: (BaCe_{0.3}Zr_{0.5}Y_{0.2}O₃ on calcined NiO—BaCe_{0.3}Zr_{0.5}Y_{0.2}O₃) layered with an Ag anode.

1A: NiO—BaCe_{0.3}Zr_{0.5}Y_{0.2}O₃ is pre-calcinated at 700° C. under pure argon for 10 hours before NiO being activated with a 25 vol. % H₂ atmosphere in argon at 700° C. for 10 hours.

1B: NiO is activated with a 25 vol. % H₂ atmosphere in argon at 700° C. for 10 hours, without a pre-calcination step.

2A: NiO—BaCe_{0.3}Zr_{0.5}Y_{0.2}O₃ is pre-calcinated at 700° C. under pure argon for 10 hours before addition of BaCe_{0.3}Zr_{0.5}Y_{0.2}O₃. Then the NiO is reduced under H₂ at 700° C. for 10 hours.

### Preparation of the Proton-Conducting Catalytic Membrane

The preparation of the reactor assembly is made as following:

25 g of the solid solution of BaCeO₃ and BaZrO₃ is then mixed and ground for 15 hours in acetone together with 37.5 g of NiO (99%-metal basis; 325 mesh powder, Alfa Aesar Ref: 12359, 250 g, CAS 1313-99-1). After a step of drying by heating at 60° C. in a furnace, the mixed oxides are mixed with polyvinyl alcohol (PVA) to favour the pressing. The mixture has a ratio of mixed oxides/PVA 1/0.075. It is then ground with Agata's mortar before being pressed uniaxially at 40 kN in the shape of a disc (disc diameter: 20 mm). Optionally, the cathode is calcined at 700° C. for 10 h.

Then, the electrolytes are coated on the cathode by using screen-printing technique using a 21-mesh to obtain a porous cathode with an electrolyte layer. The support is a pressed powder transformed into a disk. In membranes, the screen-printing procedure consists of squeezing the slurry “ink” (electrolyte) to pass through a 21-mesh screen to print it on the cathode. After drying the layer (temperature of 80° C.), it is possible to deposit extra layers. The typical thickness of the obtained layer is around 30 μm. After that, the different layers and cathode are sintered at 1565° C. temperature to obtain an efficient attachment between the cathode and the electrolyte and to afford a sintered porous cathode with an electrolyte layer.

Prior to be used, the NiO species have been reduced under hydrogen to form Nit. This activation step is carried out in a 25 vol. % H₂ atmosphere in argon at 700° C. for 10 hours, optionally with a pre-calcination at 700° C. under pure argon for 10 hours before the activation step.

Then, an anodic layer is coated on the electrolyte layer of the sintered porous cathode.

The anodic layer is an anode comprising Cu, with channels and a dehydrogenation catalyst comprised within said channels.

The sintered porous cathode with an electrolyte layer is covered with copper, using ion-sputtering. The ion-sputtering conditions involve a Pfeiffer Classic 250 deposition system equipped with two RF (13.56 MHz) power sources, each capable of delivering up to 25 W of power. The system utilizes a Cu target for the deposition process and operates at room temperature. The deposition is carried out under a pure Ar atmosphere with a pressure range of 2.6. 10⁻² to 7.4. 10⁻² mbar.

A channeled support in copper comprising a dehydrogenation catalyst is then prepared by 3D printing of the anode. The 3D printer is a Markeforged MetalX printer using a Bound Powder Filament made of copper (90%) and a polymer (10%). The 3D printing allows to make linear channels. The obtained support is then calcined to remove the polymer at about 200° C. and is sintered under Ar at about 900° C. for 72 h. Then, the channels of the support are filled by hands with 1.25 g of catalyst powder, namely of PtSnEu support on Al₂O₃ (0.5 wt. % of Pt, 2 wt. % of Eu and 3 wt. % of Sn).

The sintered porous cathode with an electrolyte layer covered with copper is thus placed on top of the channeled anode comprising the dehydrogenation catalyst and sealed with either GL1734 glass sealing paste (commercially available at Mo-Sci Corporation) or GM31107 glass sealant tape (commercially available at Schott AG) to obtain a proton-conducting catalytic membrane. The glass sealing paste is activated at 620° C. for 4 hours, while the glass sealant tape is activated at 700° C. for 2 hours.

The proton-conducting catalytic membrane is then placed into a reactor housing in Inconel alloy.

Hydrogen Extraction with the Proton-Conducting Catalytic Membrane

The present disclosure introduces a reactor comprising an electrochemical cell based on protonic-conducting materials to extract the generated H₂ and shift the equilibrium to the propane dehydrogenation reaction.

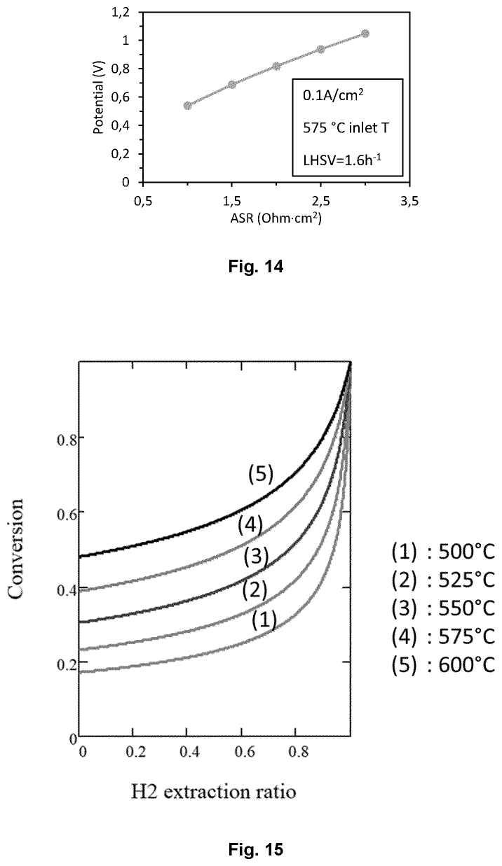

The graph displayed in FIG. 14 shows the evolution of the electrical potential in function of the area specific resistance. The best operating cell potential is about 0.5 V and about 0.6 V, although it could be of 1.0 V or even more. However, the maximum value not to cross is 1.8 V, since above this limit, the proton-conducting catalytic membrane will be damaged.

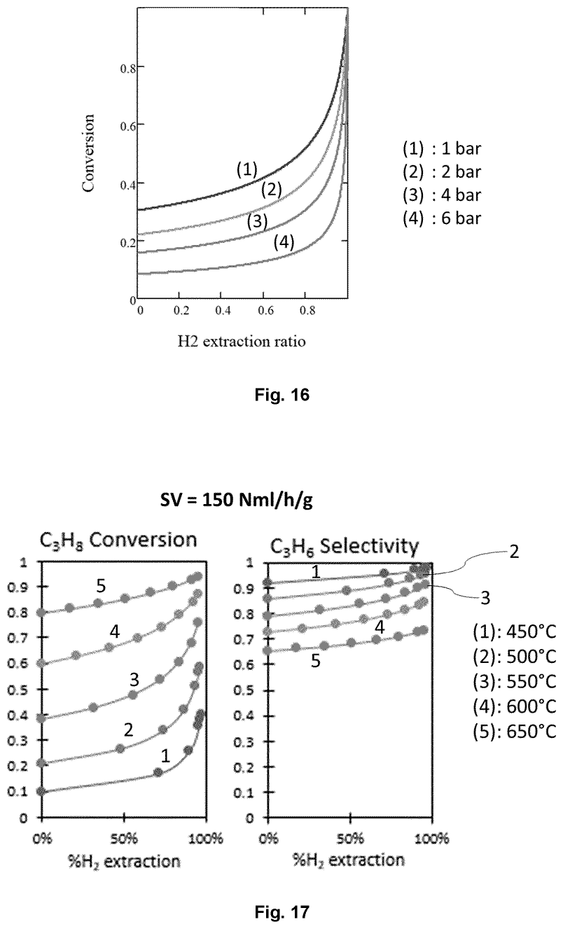

The elemental model represented in FIG. 15 (in function of the temperature) and in FIG. 16 (in function of the pressure) shows that high current density, and therefore H₂ extraction, is required to achieve high propane conversions: at 550° C., the propane equilibrium conversion reaches around 30% for a conventional reactor where no extraction is occurring. The H₂ extraction led to clear conversion improvement, notably an extraction of 80% of the H₂ generated is required to achieve conversions higher than 50%.

The H₂ extraction is modelled by considering the Faraday Law:

$${F}_{{H}_{2} e x t r a c t e d} = \frac{i}{z \cdot F} \cdot {M}_{{H}_{2}} \qquad ( 3 )$$

wherein the F_{H}_{₂}_{extracted} is the mass flux of the H₂ extracted for the H₂ extraction boundary, the i is the current density, the z is the exchanged electrons (two for the protonic exchange), F is the Faraday constant (96485 C/mol) and M_{H}_{₂} is the H₂ molecular weight. Other lateral walls were assigned a no-slip boundary condition.

CFD simulations have been performed to study the different parameters affecting the PDH reaction and to optimize the reaction performance. To maximize the membrane surface area to catalyst volume ratio and improve the fluid dynamic regime, micro-channels have been placed in the reaction chamber, namely within the andoe of the proton-conducting catalytic membrane of the present disclosure. Those channels are either rectangular or flared depth.

### One or More Rectangular Channels in the Anode

The initial geometry considered in this study consists of a series of rectangular microchannels arranged in parallel, with a length of 50 mm and a surface area of the inlet being equal to the surface area of the outlet (e.g., 25 mm²), as shown by FIG. 5. These channels have been filled with 1.25 g of catalyst (i.e., PtSnEu supported on Al₂O₃) and equipped with a selective membrane to remove H₂ on the top surface.

Table 1 shows that the H₂ is evacuated from the membrane, subsequently leading to an increase of propane conversion by comparison with a dehydrogenation reactor devoid of the proton-conducting catalytic membrane of the disclosure. The results of table 1 have been simulated by CFD at 550° C. and at 0.1 MPa.

TABLE 1
Simulation by CFD of the propane conversion, using a
proton-conducting catalytic membrane with one rectangular
channel within the anode, as shown on FIG. 6.

| | | Space | Maximum | Propane |
|---|---|---|---|---|
| | Feed flow F | Velocity SV | current density j | conversion |
| | (Nml/min) | (Nml/h/g) | (A/cm²) | (%) |
| #1 | 17.4 | 750 | 0.45 | 52 |
| #2 | 17.4 | 300 | 0.54 | 60 |
| #3 | 8.7 | 750 | 0.25 | 55 |
| #4 | 8.7 | 150 | 0.33 | 72 |

The maximum current density can be seen as the maximum hydrogen formed. As less propane is sent to the catalyst in experiment #3 compared to experiment #1, less hydrogen is formed and then less current is needed to extract this hydrogen. However, at similar conditions (same feed flow and same space velocity, a lower current density (as in experiment #1 versus experiment #2, as in experiment #3 versus experiment #4) does not allow a maximum extraction, meaning that the conversion is decreasing.

A lower feed flow allows generally to increase the propane conversion, since the catalyst has time to adsorb and activate more propane.

### One or More Flared Depth Channels in the Anode

However, an optimized geometry study showed that the optimal extraction is performed having a flared depth channel as shown in FIG. 7, namely with a surface area of the outlet (e.g., 25 mm²) being larger than the surface area of the inlet (e.g., 10 mm²). In this experiment, the length of the channel has been fixed to 50 mm. These channels have been filled with 1.25 g of catalyst (i.e., PtSnEu supported on Al₂O₃)

Table 2 summarizes the results using a dehydrogenation reactor having an outlet with a surface area of 25 mm² and an inlet with a surface area of 10 mm². These results have been simulated at a pressure of 0.1 MPa and with a feed flow F of 8.7 Nml/min.

TABLE 2
Simulation by CFD of the propane conversion using a
proton-conducting catalytic membrane with one flared
depth channel within the anode, as shown on FIG. 7.

| | | | Maximum | | |
|---|---|---|---|---|---|
| | Space | | current | Propane | Propylene |
| | Velocity SV | Temperature | density j | conversion | selectivity |
| | (Nml/h/g) | (° C.) | (A/cm²) | (%) | (%) |
| #5 | 750 | 450 | 0.13 | 29 | 99 |
| #6 | 750 | 500 | 0.19 | 44 | 98 |
| #7 | 750 | 550 | 0.25 | 57 | 96 |
| #8 | 750 | 600 | 0.28 | 57 | 91 |
| #9 | 750 | 650 | 0.28 | 73 | 80 |
| #10 | 150 | 450 | 0.17 | 40 | 98 |
| #11 | 150 | 500 | 0.24 | 58 | 96 |
| #12 | 150 | 550 | 0.30 | 76 | 91 |
| #13 | 150 | 600 | 0.33 | 87 | 84 |
| #14 | 150 | 650 | 0.32 | 94 | 73 |

It has thus been demonstrated that lower temperatures lead to lower propane conversion and higher propylene selectivity.

It has also been demonstrated that lower space velocity leads to higher propane conversion but lower propylene selectivity and thus more cracking into ethane and methane.

### Improvement Over Configuration Devoid of H₂ Extraction

Table 3 indicates the improvement of using a flared depth configuration, in comparison of a proton-conducting catalytic membrane using a rectangular channel configuration or without any proton-conducting catalytic membrane. The simulation has been made at a space velocity SV of 750 Nml/h/g.

TABLE 3
Simulation by CFD of the propane conversion and the hydrogen mole
fraction at the oulet of the proton-conducting catalytic membrane.

| | | Feed | | | Average |
|---|---|---|---|---|---|
| | | flow F | current | Propane | H₂ mole |
| | | (Nml/ | density | conver- | fraction at |
| | | min) | (A/cm²) | sion (%) | the oultet |
| #15 | no extraction | 8.7 | 0 | 28.45 | 0.176 |
| | | 17.4 | 0 | 28.70 | 0.180 |
| #16 | H₂ extraction using | 8.7 | 0.25 | 48.80 | 0.052 |
| | rectangular channels | 17.4 | 0.25 | 47.18 | 0.066 |
| #17 | H₂ extraction using | 8.7 | 0.25 | 54.57 | 0.094 |
| | flared depth channels | 17.4 | 0.25 | 51.51 | 0.051 |

It can be highlighted here that on the outlet side of the dehydrogenation reactor, the conversion of propane is reaching at least 50% when flared depth channels are present within the anode, while for a similar reactor devoid of the proton-conducting catalytic membrane of the disclosure, the conversion of propane is inferior to 30%.

### Dehydrogenation Conditions

Different dehydrogenating conditions including current intensities, feed flows and/or space velocities have been simulated as indicated in table 4 (at constant temperature) and in table 5 (at constant feed flow).

It is noted that when the dehydrogenating step is carried out at 550° C., acceptable side reactions occurred (>95% selectivity).

TABLE 4
Dehydrogenating step carried out at 550° C.

| | Optimum current | | Space | Propane |
|---|---|---|---|---|
| | intensities | Feed flows | velocities | conversion |
| #18 | 0.25 A/cm² | 8.7 Nml/min | 750 Nml/h/g | 55% |
| #19 | 0.33 A/cm² | 8.7 Nml/min | 150 Nml/h/g | 72% |
| #20 | 0.45 A/cm² | 17.4 Nml/min | 750 Nml/h/g | 52% |
| #21 | 0.54 A/cm² | 17.4 Nml/min | 300 Nml/h/g | 60% |

The highest quantity of formed H₂ anticipated by CFD simulations has been obtained without side reactions, at a feed flow of 17.4 NmL/min; space velocity of 300 NmL/H/g and current intensity of 0.54 A/cm² (see experiment 21). In such conditions, 10.44 Nml of H₂ formed which 5 is below the extraction potential of the membrane for a single channel of 2.5 cm² prepared as an example: H₂ extraction of 5 NmL·min⁻¹·cm⁻² meaning 12.5 NmL/min. It showed that the extraction level reached by the disclosure is satisfying. Indeed, through perfect gas law, as it is normal flow rate then the normal volume is as follows 1 mmol=22.4 ml. Given that the feed flow is 17.4 Nml/min, the number of mole is amounting to 0.78 mmol. As the conversion is 60% (see experiment 21), the volume of H₂ formed is 10.44 Nml.

Under the propane dehydrogenation conditions, the propane can be oxidized into propylene but also other compounds are formed, such as ethylene, ethane and methane. The selectivities were assessed in accordance with the conditions given in table 5.

TABLE 5
Dehydrogenating step carried out at a feed flow of 8.7 Nml/min

| | | Optimum | | | | |
|---|---|---|---|---|---|---|
| | | current | Space | Propane | Propylene | Propylene |
| | Temperature | intensities | velocities | conversion | selectivity | yield |
| #22 | 450° C. | 0.13 A/cm² | 750 | 29% | 99% | 28.7% |
| | | | Nml/h/g | | | |
| #23 | 450° C. | 0.17 A/cm² | 150 | 40% | 98% | 39.2% |
| | | | Nml/h/g | | | |
| #24 | 500° C. | 0.19 A/cm² | 750 | 44% | 98% | 43.1% |
| | | | Nml/h/g | | | |
| #25 | 550° C. | 0.24 A/cm² | 150 | 58% | 96% | 55.7% |
| | | | Nml/h/g | | | |
| #26 | 550° C. | 0.25 A/cm² | 750 | 57% | 96% | 54.7% |
| | | | Nml/h/g | | | |
| #27 | 550° C. | 0.30 A/cm² | 150 | 76% | 91% | 69.2% |
| | | | Nml/h/g | | | |
| #28 | 600° C. | 0.28 A/cm² | 750 | 57% | 91% | 51.9% |
| | | | Nml/h/g | | | |
| #29 | 600° C. | 0.33 A/cm² | 150 | 87% | 84% | 73.1% |
| | | | Nml/h/g | | | |
| #30 | 650° C. | 0.28 A/cm² | 750 | 73% | 80% | 58.4% |
| | | | Nml/h/g | | | |
| #31 | 650° C. | 0.32 A/cm² | 150 | 94% | 73% | 68.6% |
| | | | Nml/h/g | | | |

Experiment #29 provides a yield in propylene of 73.1%.

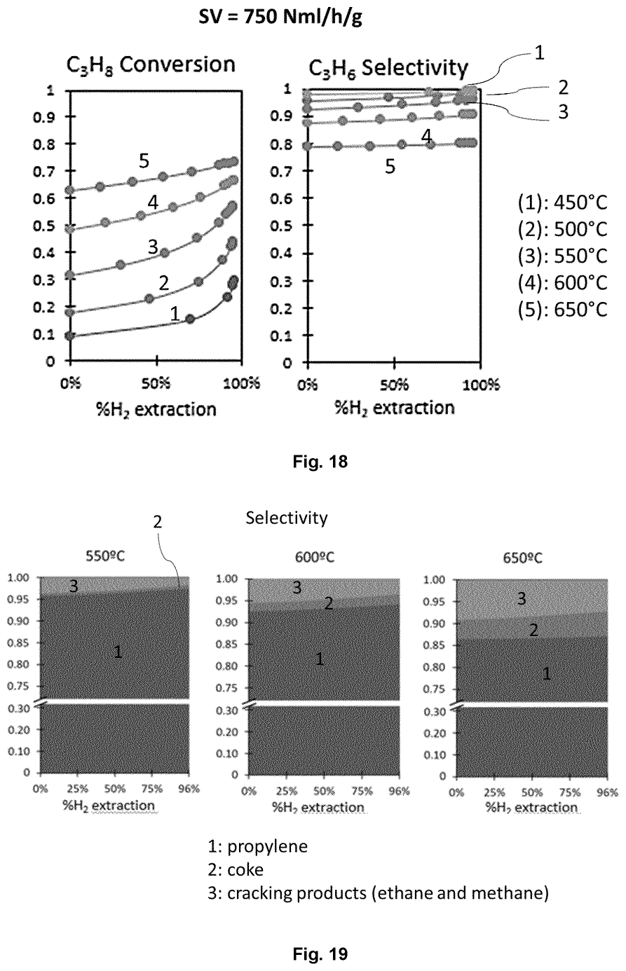

Furthermore, the CFD simulation results have been plotted as propane conversion and propylene selectivity against the H₂ extraction ratio, for different temperatures and space velocities (SV). The flowrate has been set to 8.7 Nml/min, while the current density varied from 0 (no H₂ extraction) to a maximum value, which is indicated in table 5. This maximum value corresponds to the point where the H₂ molar fraction near the outlet becomes “negative”. FIG. 17 shows the results for a space velocity of 150 Nml/h/g and FIG. 18 shows the results for a space velocity of 750 Nml/h/g.

As expected, H₂ extraction has a clear impact on conversion at relatively low temperatures. Indeed, below 600° C. the propane conversion could increase by 20-40% depending on the conditions. On the contrary, at higher temperatures (650° C.), the H₂ extraction has little impact on the conversion since the system becomes kinetically limited. However, a higher temperature leads to larger conversion values, but selectivity to propylene decreases.

Comparing the space velocities, for SV=150 (i.e., higher catalyst loading), higher conversion values are obtained and H₂ extraction affects the conversion a little bit more since kinetic limitations are overcome. But increasing the catalyst loading has an important disadvantage: the activation of the side reactions leads to a lower propylene selectivity.

At SV of 150, an optimum was found between propylene selectivity and propane conversion at 550° C., propylene selectivity could be over 90% and propane conversion over 50% if over 90% of the H₂ produced is extracted (see #32 in table 6).

TABLE 6
Dehydrogenating step carried out in the best conditions

| | | Current | Feed | Space | Propane | Propylene |
|---|---|---|---|---|---|---|
| | Temperature | intensities | flow | velocities | conversion | selectivity |
| #32 | 550° C. | 0.30 A/cm² | 8.7 Nml/min | 150 Nml/h/g | 76% | 91% |

Experiment #32 provides a yield in propylene of 69.2%.

### Preventing Coke Formation at Temperature Superior to 550° C.

FIG. 19 shows that the formation of coke from propylene is occurring at temperature above 550° C., reducing thus the selectivity into propylene. The formation of coke tends to increase at higher temperature, for example up to 7% of coke at a temperature of 650° C. and at an H₂ extraction of 96%. The coking generates a loss of selectivity.

The coke deposition and further suppression were modeled by CFD simulations. The two solutions envisioned were water co-feeding and/or water co-feeding with use of a co-ionic membrane, as shown in table 7.

TABLE 7
Suppression of coke deposition by water co-feeding or water co-feeding along
with use of a co-ionic membrane. Experiments were carried out at 575° C., with
a current intensity of 0.1 A/cm² and a time on stream (TOS) of 100 h.

| | | No steam | | | Addition of co-ionic | |
|---|---|---|---|---|---|---|
| | | Coke | Water-co-feeding | | membrane | |
| | Cross- | deposition | H₂O molar | Coke | H₂O molar | Coke |
| | section | (mg_{Coke}/ | fraction | suppression | fraction | suppression |
| | (cm) | g_{cat}) | (%) | (%) | (%) | (%) |
| #33 | 0.10 | 0.37 | 2.76 | 100 | 0.1 | 20.5 |
| #34 | 1.25 | 1.14 | 2.28 | 98.3 | 0.6 | 75.9 |
| #35 | 2.50 | 1.27 | 2.07 | 98.1 | 1.2 | 92.8 |
| #36 | 3.75 | 1.32 | 1.95 | 98.5 | 1.7 | 97.3 |
| #37 | 4.90 | 1.35 | 1.86 | 98.8 | 2.1 | 98.7 |

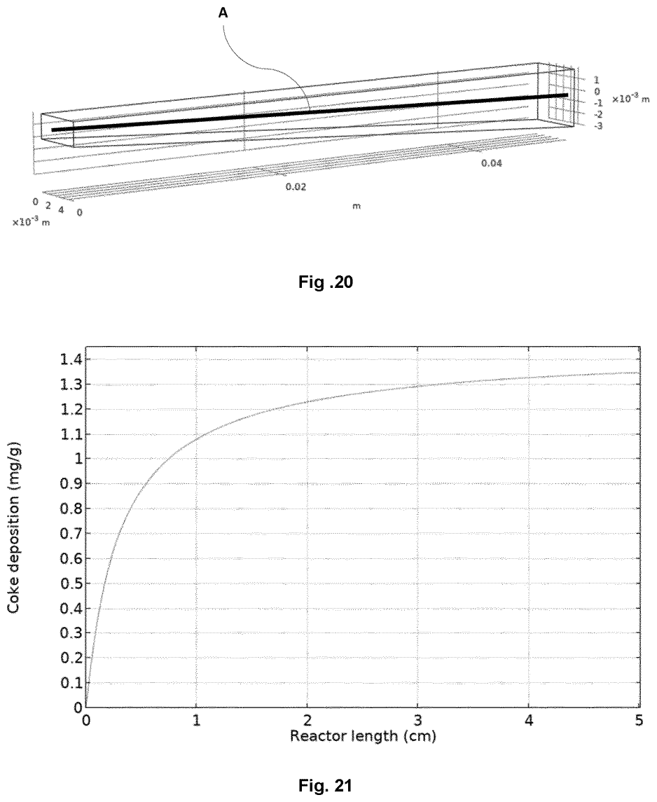

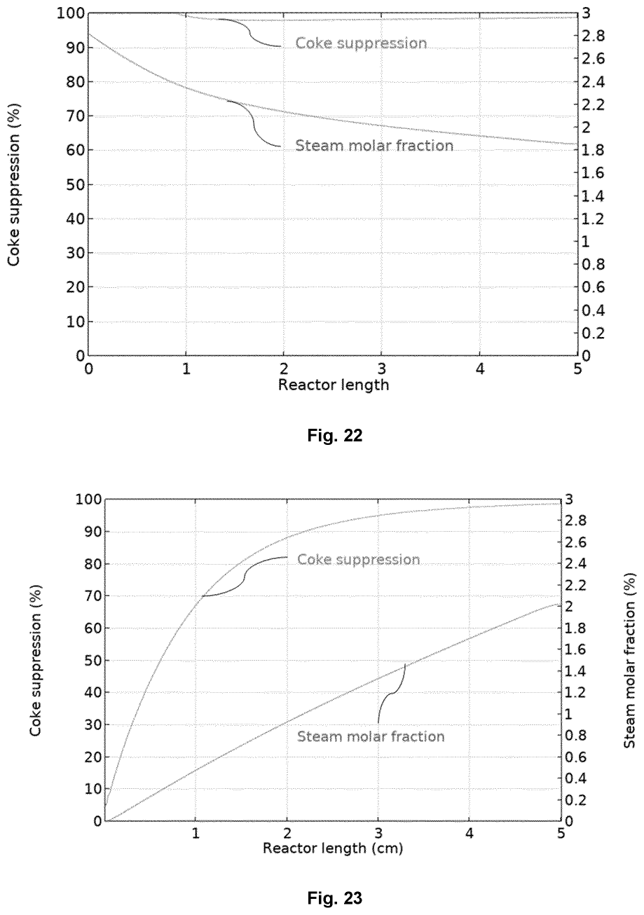

FIG. 20 shows a flared depth channels in which the line A was modelled into graphs showing the evolution of the amount of coke within the channels. Thus, FIG. 21 shows the evolution of the coke deposition in function of the reactor length in the absence of steam, while FIG. 22 shows the evolution of the coke suppression in function of the reactor length when water is co-fed with the propane and when the reactor employs a proton-conducting catalytic membrane in accordance with the disclosure and FIG. 23 shows the evolution of the coke suppression in function of the reactor length when water is co-fed with the propane and when the proton-conducting catalytic membrane of the reactor is a co-ionic membrane.

Both solutions showed coke efficient suppression leading to very stable propylene yield for 100 hours on stream. In contrast, the absence of steam affords a significant drop in yield after 25 hours on stream.

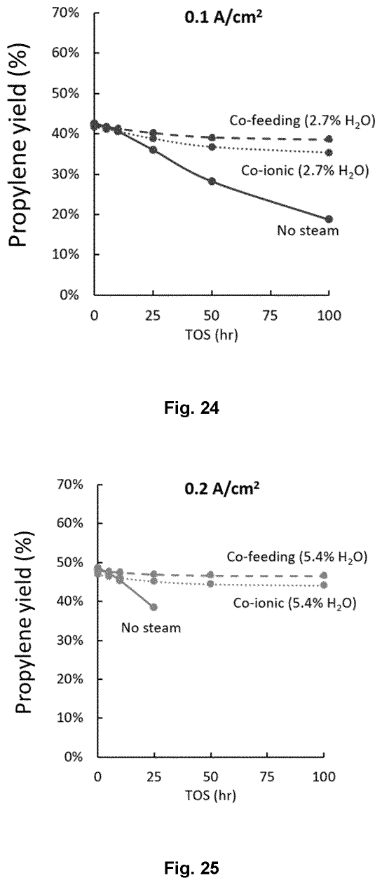

FIGS. 24 to 26 show the evolution of the propylene yield in function of the time on stream (TOS).

FIG. 24 shows that the co-feeding of water into the stream comprising propane at a molar fraction of 2.7% allows to maintain the yield into propylene as about 40% at a current density of 0.1 A/cm².

FIG. 25 shows that the co-feeding of water into the stream comprising propane at a molar fraction of 5.4% allows to maintain the yield into propylene as about 50% at a current density of 0.2 A/cm².

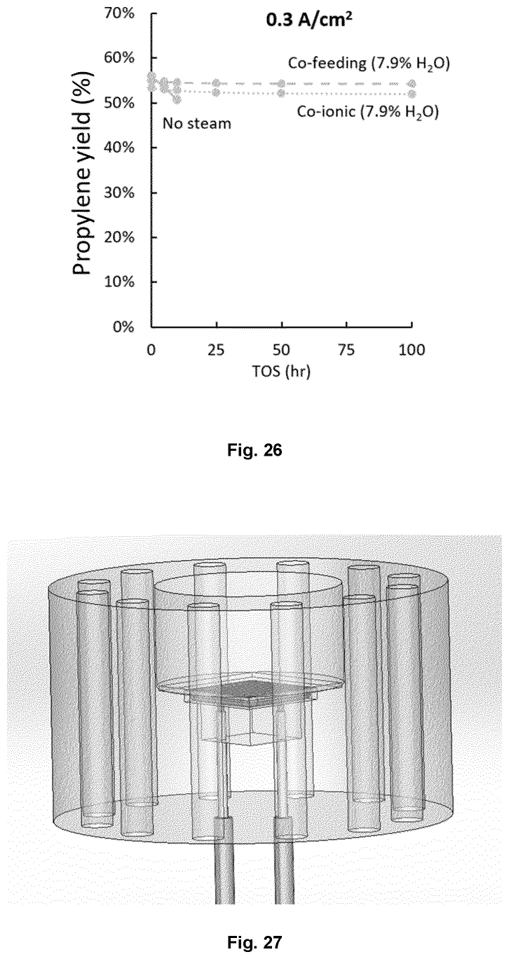

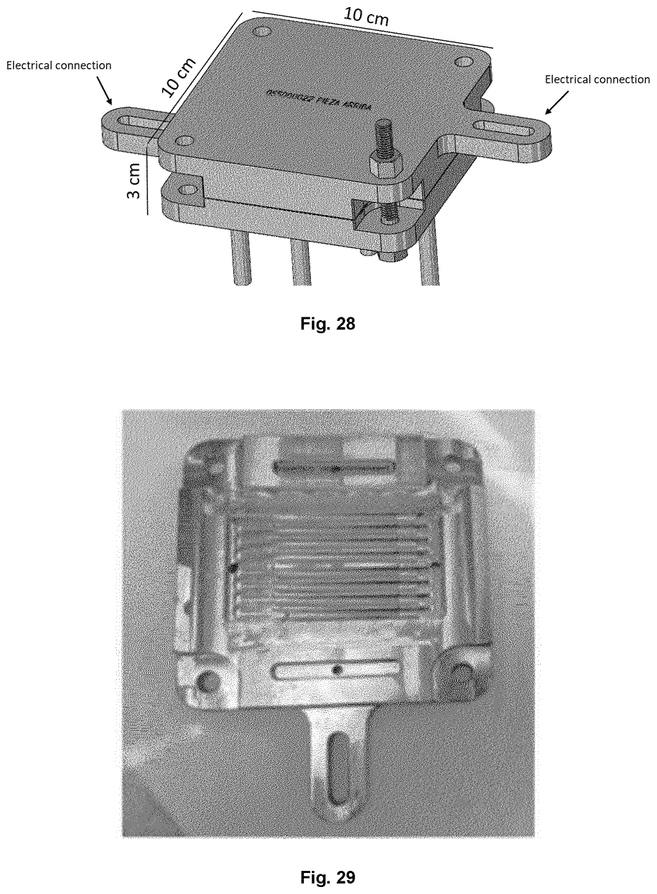

FIG. 26 shows that the co-feeding of water into the stream comprising propane at a molar fraction of 7.9% allows to maintain the yield into propylene as about 55% at a current density of 0.3 A/cm².

FIG. 27 is a view of the reactor assembly in which the anode is made of copper. An external steel housing is shown. The copper anode is for example incorporated within the external steel housing. The scheme shows the channels into the copper anode, that have been made using 3D printing. For example, the reactor assembly has a diameter of 17 cm and a height of 10 cm. The electrical connection to the copper anode must be made through the external steel housing.

Proton-Conducting Catalytic Membrane with an Anode in Steel

The preparation of the reactor assembly (see FIG. 26) is made as following:

25 g of the solid solution of BaCeO₃ and BaZrO₃ is mixed and ground for 15 hours in acetone together with 37.5 g of NiO (99%-metal basis; 325 mesh powder, Alfa Aesar Ref: 12359, 250 g, CAS 1313-99-1). After a step of drying by heating at 60° C. in a furnace, the mixed oxides are mixed with polyvinyl alcohol (PVA) to favour the pressing. The mixture has a ratio of mixed oxides/PVA 1/0.075. It is then ground with Agata's mortar before being pressed uniaxially at 40 kN in the shape of a disc (disc diameter: 20 mm). Optionally, the cathode is calcined at 700° C. for 10 hours.

Then, the electrolytes are coated on the cathode by using screen-printing technique using a 21-mesh to obtain a porous cathode with an electrolyte layer. The support is a pressed powder transformed into a disk. In membranes, the screen-printing procedure consists of squeezing the slurry “ink” (electrolyte) to pass through a 21-mesh screen to print it on the cathode. After drying the layer (temperature of 80° C.), it is possible to deposit extra layers. The typical thickness of the obtained layer is around 30 μm. After that, the different layers and cathode are sintered at 1565° C. temperature to obtain an efficient attachment between the cathode and the electrolyte and to afford a sintered porous cathode with an electrolyte layer.

Prior to be used, the NiO species have been reduced under hydrogen to form Nit. This activation step is carried out in a 25 vol. % H₂ atmosphere in argon at 700° C. for 10 hours, optionally with a pre-calcination at 700° C. under pure argon for 10 hours before the activation step.

Then, an anodic layer is coated on the electrolyte layer of the sintered porous cathode.

The anodic layer is an anode of stainless steel, with channels and a dehydrogenation catalyst comprised within said channels.

The sintered porous cathode with an electrolyte layer is covered with copper, using ion-sputtering. The ion-sputtering conditions involve a Pfeiffer Classic 250 deposition system equipped with two RF (13.56 MHz) power sources, each capable of delivering up to 25 W of power. The system utilizes a Cu target for the deposition process and operates at room temperature. The deposition is carried out under a pure Ar atmosphere with a pressure range of 2.6. 10-2 to 7.4. 10-2 mbar.

A channeled support in steel comprising a dehydrogenation catalyst is then prepared by deep drawing or machining. The fact that deep drawing or machining can be used to prepared the channeled support is advantageous in the sense that it avoids the use of the 3D printing technique. A steel plate and quartz wool are used around the catalyst channels to avoid the catalyst to move. GM31107 glass sealant tape (commercially available at Schott AG) is used to sealed the steel plate to the steel support. The glass sealing tape is activated at 700° C. for 2 hours.

Then, the channels of the support are filled by hands with 1.25 g of catalyst powder, namely of PtSnEu support on Al₂O₃ (0.5 wt. % of Pt, 2 wt. % of Eu and 3 wt. % of Sn).

The sintered porous cathode with an electrolyte layer covered with copper is thus placed on top of the channeled support comprising the dehydrogenation catalyst and sealed with either GL1734 glass sealing paste (commercially available at Mo-Sci Corporation) or Cotronics Resbond® 908 paste (i.e., an alumina-based bonding ceramic cement) to obtain a proton-conducting catalytic membrane. The glass sealing paste is activated at 620° C. for 4 hours, while the Cotronics tape is cured 24 h at room temperature with applied weight.

Finally, an upper steel piece with channels is added to the channeled support and proton-conducting catalytic membrane. Then both steel pieces are sealed together using either GL1734 glass sealing paste (commercially available at Mo-Sci Corporation) or Cotronics Resbond® 908 paste (i.e., an alumina-based bonding ceramic cement) to obtain a proton-conducting catalytic membrane. The glass sealing paste is activated at 620° C. for 4 hours, while the Cotronics tape is cured 24 h at room temperature with applied weight.

A closed membrane reactor is thus obtained (see FIGS. 26 and 27). As the steel anode comprises the channels, the risks of leaks are decreased once the reactor has been completed. The shape of the steel anode allows for having electrical connections on the side of the membrane, which facilitates their access. This is advantageous in comparison with a reactor assembly with a reactor housing made of Inconel, since in this case, the electrical connections cannot be in contact with the Inconel to ensure an adequate connection.

1. A propane dehydrogenation process is characterized in that it comprises the following steps:
a) providing a stream comprising at least propane;
b) providing at least one proton-conducting catalytic membrane, each proton-conducting catalytic membrane comprising an anode, an electrolyte layer disposed on top of the anode and a porous cathode disposed on top of the electrolyte layer, wherein the anode comprises one or more channels, each of said channels comprising at least one dehydrogenation catalyst; wherein said one or more channels are flared depth channels or rectangular channels, and wherein said one or more channels are made in copper or steel;
c) feeding within the anode of said one or more proton-conducting catalytic membranes under propane dehydrogenation conditions the stream provided at step (a);
d) recovering a first effluent comprising at least propylene;
e) optionally, recovering a second effluent comprising hydrogen.

2. The propane dehydrogenation process according to claim 1 is characterized in that the propape dehydrogenation conditions of step (c) comprise a temperature ranging between 425° C. and 575° C.

3. The propane dehydrogenation process according to claim 1 is characterized in that the propane dehydrogenation conditions comprise a space velocity of at least 150 Nml/h/g.

4. The propane dehydrogenation process according to claim 1 is characterized in that it comprises a step of providing steam into the stream provided at step (a) in a pulsed manner.

5. The propane dehydrogenation process according to claim 1 is characterized in that the stream provided at step (a) further comprises steam in an amount ranging between 1 vol. % and 15 vol. % of the total volume of the stream provided at step (a).

6. The propane dehydrogenation process according to claim 1 is characterized in that the one or more proton-conducting catalytic membranes provided at step (b) are one or more co-ionic catalytic membranes.

7. A polypropylene manufacturing process comprising the following steps:
a) providing a first propylene stream, said first propylene stream optionally comprising one or more solvents;
b) polymerizing the first propylene stream under polymerization conditions to form an effluent comprising polypropylene and at least propylene and propane and optionally the one or more solvents if any;
c) separating the effluent to generate a first stream comprising polypropylene and a second stream comprising at least propylene and propane, and optionally a third stream comprising the one or more solvents if any;
d) optionally, recycling the third stream if any;
f) splitting the second stream so as to produce an overhead comprising propylene and a purge stream, said purge stream being a propane-rich stream further comprising propylene;
g) optionally, recycling the overhead into the first propylene stream provided at step (a);
wherein said process is characterized in that it further comprises a first dehydrogenating step (h) and/or a second dehydrogenating step (e);
wherein said first dehydrogenating step (h) is the step of dehydrogenating the purge stream produced at step (f) to enrich in propylene the purge stream;
wherein said second dehydrogenating step (e) is carried out after the separation step (c) and before the spliiting step (f) and is the step of dehydrogenating the second stream generated at step (c) to enrich in propylene the second stream; and wherein said first and second dehydrogenating steps are each a propane dehydrogation step as defined in accordance with claim 1.

8. The polypropylene manufacturing process according to claim 7 is characterized in that said process further comprises a first dehydrogenating step (h).

9. The polypropylene manufacturing process according to claim 7 is characterized in that during the first dehydrogenating step (h) and/or during the second dehydrogenating step (e), a step of recovering a hydrogen effluent is carried out.

10. The polypropylene manufacturing process according to claim 7 is characterized in that the propane dehydrogenation conditions of the first dehydrogenating step (h) and/or the second dehydrogenating step (e) comprise providing an electrical current between the anode and the porous cathode of the one or more proton-conducting catalytic membranes.

11. The polypropylene manufacturing process according to claim 10 is characterized in that said electrical current has a density ranging between 0.10 A/cm² and 0.75 A/cm² as measured by ampere meter/power supply instrument and divided by membrane surface.

12. The polypropylene manufacturing process according to claim 7 is characterized in that the purge stream has during the first dehydrogenating step (h) a feed flow ranging between 0.5 T/h and 5 T/h as measured by a flowmeter.

13. The polypropylene manufacturing process according to claim 7 is characterized in that the second stream has during the second dehydrogenating step (e) a feed flow ranging between 20 T/h and 40 T/h as measured by a flowmeter.

14. The polypropylene manufacturing process according to claim 7 is characterized in that the second stream comprises after the second dehydrogenating step (e) at least 1.5 times more propylene than before said second dehydrogenating step (e).

15. The polypropylene manufacturing process according to claim 7 is characterized in that the purge stream comprises after the first dehydrogenating step (h) at least 1.5 times more propylene than before said first dehydrogenating step (h).

16. An installation to conduct a polypropylene manufacturing process as defined in accordance with claim 7, said installation comprising one polymerization zone (1) with one polymerization reactor (9) and one separation zone (3) downstream of said polymerization zone (1), said separation zone (3) comprising a polymer separation apparatus (21) and a splitter (11) with a bottom outlet (13), wherein said installation further comprises a first line (5) between said polymerization reactor (9) and said polymer separation apparatus (21), said installation is characterized in that it comprises one or more first dehydrogenation reactors (15) arranged downstream of the bottom outlet (13) of the splitter and/or one or more second dehydrogenation reactors (16) arranged upstream of the splitter (11), each first and second dehydrogenation reactor (15, 16) comprising at least one proton-conducting catalytic membrane, each proton-conducting catalytic membrane comprising an anode, an electrolyte layer disposed on top of the sanode and a porous cathode disposed on top of the electrolyte layer, and wherein the anode comprises one or more channels, each of said channels comprising at least one dehydrogenation catalyst, wherein said one or more channels are flared depth channels or rectangular channels, and wherein said one or more channels are made in copper or steel.

17. The installation according to claim 16 is characterized in that said installation comprises one or more first dehydrogation reactors (15) arranged downstream of the bottom outlet (13) of the splitter.

18. The installation according to claim 16 is characterized in that said installation further comprises one scrubber (17) arranged upstream of each first and/or second dehydrogenation reactor (15).

19. The installation according to claim 16 is characterized in that the splitter (11) has a top outlet, and the installation further comprises a recycling line (29) between the top outlet of the splitter (11) and the polymerization reactor (9).

20. The installation according to claim 19 is characterized in that a dryer system (31) is upstream the polymerization reactor (9) on the recycling line (29).

21. (canceled)
